(19) United States
(12) Patent Application Publication
(10) Pub. No.: US 2009/0258883 A1
(43) Pub. Date: Oct. 15, 2009

(54) 1H-Indole-Pyridinecarboxamide and 1H-Indole-Piperidinecarboxamide Compounds

(75) Inventors: Jean-Daniel Brion, Saint Leulla Foret (FR); Fabrice Bintein, Velizy (FR); Rodolphe Razet, Ridiesheim (FR); Patrick Razon, Paris (FR); Zafiarisoa Dolor Renko, Les Ulis (FR); Eric Levoirier, Choisy Le Roi (FR); Jean-Francois Pujol, Paris (FR); Dinah Weissmann, Paris (FR); Alain Le Ridant, Neuilly Sur Seine (FR); Catherine Harpey, Paris (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignees: LES LABORATOIRES SERVIER, COURBEVOIE CEDEX (FR); CENTRE NATIONAL DE LA RECHERCHE SCIENTIFIQUE, PARIS CEDEX (FR); UNIVERSITE PARIS SUD, ORSAY CEDEX (FR)

(21) Appl. No.: 11/988,424

(22) PCT Filed: Jul. 6, 2006
(86) PCT No.: PCT/FR2006/001610

§ 371 Date: Jan. 4, 2008

(30) Foreign Application Priority Data
Jul. 7, 2005 (FR) 0507225

(51) Int. Cl. A61K 31/496; C07D 401/12; A61K 31/454; A61P 25/22; A61P 25/16; A61P 25/24; A61P 25/28; A61K 31/4439; C07D 401/14

(52) U.S. Cl. 514/253.09; 546/201; 514/323; 514/339; 546/277.4; 544/364

(57) ABSTRACT

Compounds of formula (I):

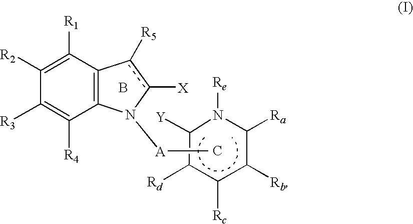

wherein:
A represents a divalent radical:

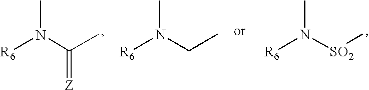

wherein:
Z represents an oxygen atom or a sulphur atom,
R₆ represents a hydrogen atom, an alkyl, alkenyl, arylalkyl or polyhaloalkyl group or a substituted, linear or branched alkyl chain, [symbol]
represents a single bond or a double bond,
R₁, R₂, R₃ and R₄ represent a hydrogen or halogen atom,
an alkyl, alkoxy, hydroxy, cyano, nitro, polyhaloalkyl or optionally substituted amino group, or a linear or branched alkyl chain substituted by one or more groups,
R₅ represents a hydrogen atom or an alkyl, aminoalkyl or hydroxyalkyl group,
X and Y represent a hydrogen atom or an alkyl group,
Rₐ, R_{b}, R_{c} and R_{d} represent a hydrogen or halogen atom, an alkyl, hydroxy, alkoxy, cyano, nitro, polyhaloalkyl, optionally substituted amino group, or a substituted, linear or branched alkyl chain,
Rₑ represents a hydrogen atom or an alkyl, arylalkyl or alkenyl group or a substituted, linear or branched alkyl chain,
   their enantiomers, diastereoisomers, and N-oxides, and also addition salts thereof with a pharmaceutically acceptable acid or base.

The present invention relates to new 1H-indole-pyridinecarboxamide and 1H-indole-piperidinecarboxamide compounds, to a process for their preparation and to pharmaceutical compositions containing them.

The literature provides numerous examples of compounds exhibiting an eburnane structure, this being the case especially with the patent specification U.S. Pat. No. 3,454,583, which deals with vincamine(methyl (3α,14β,16α)-(14,15-dihydro-14-hydroxy-eburnamenine-14-carboxylate) and compounds thereof with regard to their vasodilatory properties. The Patent Applications FR 2 433 528 and FR 2 381 048 present new 20,21-dinoreburn-amenine compounds and the Patent Application EP 0 287 468 presents new 17-aza-20,21-dinoreburnamenine compounds. The Patent Application EP 0 658 557 describes eburnane compounds modified in the 14- and 15-positions of the eburnane skeleton. The Patent Application EP 0 563 916 describes 1H-indole-cyclohexanecarboxamide compounds.

Besides the fact that they are new, the compounds of the present invention have very valuable pharmacological properties. In particular, they have been found to be powerful selective or non-selective tyrosine hydroxylase inducers.

More specifically, the present invention relates to compounds of formula (I):

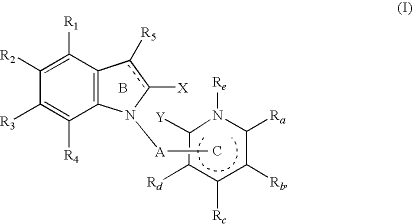

wherein:
A represents a divalent radical

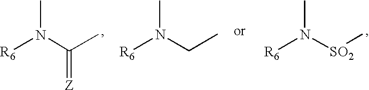

wherein:
Z represents an oxygen atom or sulphur atom,
R₆ represents:
a hydrogen atom,
a linear or branched (C₁-C₆)alkyl group, C(O)-AA wherein AA represents an amino acid radical, a linear or branched (C₁-C₆)alkoxy-carbonyl group, CHR′—O—C(O)—R″ wherein R′ represents a hydrogen atom or a linear or branched (C₁-C₆)alkyl group and R″ represents a linear or branched (C₁-C₆)alkyl group,
a linear or branched (C₂-C₆)alkenyl group, an aryl group, an aryl-(C₁-C₆)alkyl group in which the alkyl moiety is linear or branched, a linear or branched (C₁-C₆)polyhaloalkyl group,
or a linear or branched (C₁-C₆)alkyl chain substituted by one or more halogen atoms, one or more hydroxy groups, linear or branched (C₁-C₆)alkoxy groups, or amino groups optionally substituted by one or two identical or different, linear or branched (C₁-C₆)alkyl groups,
in the ring B
[symbol] represents a single bond or a double bond,
in the ring C
[symbol] represents a single bond or a double bond, the ring C containing, at most, only one double bond,
R₁, R₂, R₃ and R₄, which may be the same or different, each independently of the others, represent:
a hydrogen or halogen atom,
a linear or branched (C₁-C₆)alkyl group, a linear or branched (C₁-C₆)alkoxy group, a hydroxy group, a cyano group, a nitro group, a linear or branched (C₁-C₆)polyhaloalkyl group, an amino group (optionally substituted by one or two linear or branched (C₁-C₆)alkyl
and/or linear or branched (C₂-C₆)alkenyl groups, it being possible for the alkyl and alkenyl groups to be the same or different),
or a linear or branched (C₁-C₆)alkyl chain substituted by one or more halogen atoms, one or more hydroxy groups, linear or branched (C₁-C₆)alkoxy groups, or amino groups optionally substituted by one or two identical or different, linear or branched (C₁-C₆)alkyl groups,
R₅ represents:
a hydrogen atom,
a linear or branched (C₁-C₆)alkyl group, an aminoalkyl group in which the alkyl moiety is a linear or branched chain of 1 to 6 carbon atoms, or a linear or branched (C₁-C₆)-hydroxyalkyl group,
X and Y, which may be the same or different, each independently of the other, represent:
a hydrogen atom or a linear or branched (C₁-C₆)alkyl group,
Rₐ, R_{b}, R_{c} and R_{d}, which may be the same or different, each independently of the others, represent:
a hydrogen or halogen atom,
a linear or branched (C₁-C₆)alkyl group, a hydroxy group, a linear or branched (C₁-C₆)alkoxy group, a cyano group, a nitro group, a linear or branched (C₁-C₆)polyhaloalkyl group, an amino group (optionally substituted by one or two identical or different, linear or branched (C₁-C₆)alkyl groups),
or a linear or branched (C₁-C₆)alkyl chain substituted by one or more groups selected from halogen, hydroxy, linear or branched (C₁-C₆)alkoxy, and amino optionally substituted by one or two identical or different, linear or branched (C₁-C₆)alkyl groups, it being understood that when A is linked to the ring C at a carbon atom carrying one of the substituents Rₐ, R_{b}, R_{b}, R_{d} or Y and said linking carbon atom also carries a double bond, then the corresponding substituent Rₐ, R_{b}, R_{b}, R_{d} or Y is absent,
Rₑ represents:
a hydrogen atom,
a linear or branched (C₁-C₆)alkyl group; an aryl-(C₁-C₆)alkyl group in which the alkyl moiety is linear or branched; a linear or branched (C₂-C₆)alkenyl group; a linear or branched (C₂-C₆)alkynyl group; a linear or branched (C₁-C₆)alkyl chain substituted by one or more groups selected from hydroxy, amino (optionally substituted by one or two identical or different, linear or branched (C₁-C₆)alkyl groups), linear or branched (C₁-C₆)alkoxy, and NR₇R₈ wherein R₇ and R₈, together with the nitrogen atom carrying them, form an optionally substituted, 4- to 8-membered heterocycle optionally containing one or more double bonds within the heterocycle and optionally containing within the cyclic system a second hetero atom selected from an oxygen atom and a nitrogen atom; or a linear or branched (C₂-C₆)alkenyl chain substituted by the same groups as the alkyl chain or a linear or branched (C₂-C₆)alkynyl chain substituted by the same groups as the alkyl chain,
   to their enantiomers, diastereoisomers, and N-oxides, and also to addition salts thereof with a pharmaceutically acceptable acid or base,
   it being understood that:
   as an optionally substituted, 4- to 8-membered heterocycle optionally containing one or more double bonds within the heterocycle and optionally containing within the cyclic system a second hetero atom selected from an oxygen atom and a nitrogen atom, there may be mentioned, without implying any limitation, pyrrolidine, piperidine, azepane, piperazine and morpholine, those heterocycles optionally being substituted (including on the second nitrogen atom of piperazine) by one or more identical or different groups selected from linear or branched (C₁-C₆)alkyl, linear or branched (C₁-C₆)hydroxyalkyl, linear or branched (C₁-C₆)alkoxy-(C₁-C₆)alkyl, CO₂Rᵥ, CO₂—R_{w}—NRᵥR′ᵥ, CO₂—R_{w}—ORᵥ(wherein Rᵥ represents a hydrogen atom or a linear or branched (C₁-C₆)alkyl group, R′ᵥ is as defined for Rᵥ and R_{w} represents a linear or branched (C₁-C₆)alkylene chain), aryl, aryloxycarbonyl, linear or branched aryl-(C₁-C₆)alkoxy-carbonyl, optionally substituted cycloalkyl, optionally substituted cycloalkylalkyl, optionally substituted heterocycloalkyl, optionally substituted heterocycloalkylalkyl, and aminoalkyl in which the alkyl moiety is a linear or branched chain of 1 to 6 carbon atoms and the amino moiety optionally is substituted by one or two identical or different, linear or branched (C₁-C₆)alkyl groups,
   aryl means a phenyl or naphthyl group, each optionally being substituted by one or more halogen atoms, nitro, amino, linear or branched (C₁-C₆)alkyl or linear or branched (C₁-C₆)-alkoxy groups,
   cycloalkyl means a saturated, 4- to 8-membered, monocyclic group,
   cycloalkylalkyl means a cycloalkyl-alkyl group wherein the alkyl group denotes a linear or branched chain of 1 to 6 carbon atoms and the cycloalkyl group denotes a saturated, 4- to 8-membered, monocyclic group,
   heterocycloalkyl means a saturated, 4- to 8-membered, monocyclic group containing 1 or 2 hetero atoms selected from nitrogen, oxygen and sulphur,
   heterocycloalkylalkyl means a heterocycloalkyl-alkyl group wherein the alkyl group denotes a linear or branched chain of 1 to 6 carbon atoms and the heterocycloalkyl group denotes a saturated, 4- to 8-membered, monocyclic group containing 1 or 2 hetero atoms selected from nitrogen, oxygen and sulphur,
   the expression “optionally substituted” when referring to the groups cycloalkyl, cycloalkylalkyl, heterocycloalkyl and heterocycloalkylalkyl means that those groups may be substituted by one or more identical or different substituents selected from linear or branched (C₁-C₆)alkyl, linear or branched (C₁-C₆)hydroxyalkyl, linear or branched (C₁-C₆)alkoxy-(C₁-C₆)alkyl, carboxy, linear or branched (C₁-C₆)alkoxy-carbonyl and aminoalkyl in which the alkyl moiety is a linear or branched chain of 1 to 6 carbon atoms and the amino moiety optionally is substituted by one or two identical or different, linear or branched (C₁-C₆)alkyl groups,
   an amino acid radical is understood to mean the radicals alanyl, arginyl, asparaginyl, α-aspartyl, cysteinyl, α-glutamyl, glutaminyl, glycyl, histidyl, isoleucyl, leucyl, lysyl, methionyl, phenylalanyl, prolyl, seryl, threonyl, tryptophyl, tyrosyl and valyl.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid, lysine etc. . . .

Among the pharmaceutically acceptable bases there may be mentioned, without implying any limitation, sodium hydroxide, potassium hydroxide, triethylamine, tert-butylamine etc.

Preferred compounds of the invention are those wherein A represents a divalent radical

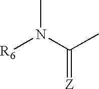

wherein R₆ is as defined for formula (I) and Z represents an oxygen atom.

According to the invention, preference is given to a hydrogen atom as the substituent R₆.

According to the invention, preference is given to a hydrogen atom, a halogen atom or a linear or branched (C₁-C₆)alkoxy group as the substituents R₁, R₂, R₃ and R₄.

According to the invention, preference is given to a hydrogen atom or a linear or branched (C₁-C₆)alkyl group as the substituent R₅.

According to the invention, preference is given to a hydrogen atom or a linear or branched (C₁-C₆)alkyl group as the substituents X and Y.

According to the invention, preference is given to a hydrogen atom as the substituents Rₐ, R_{b}, R_{c} and R_{d}.

According to the invention, preference is given to a hydrogen atom or a linear or branched (C₁-C₆)alkyl group or linear or branched (C₂-C₆)alkenyl group as the substituent Rₑ.

According to an advantageous embodiment of the invention, preferred compounds are compounds of formula (IA):

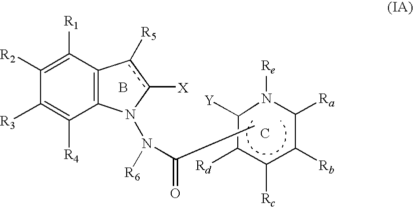

wherein [symbol] X, Y, R₁, R₂, R₃, R₄, R₅, R₆, Rₐ, R_{b}, R_{c}, R_{d} and Rₑ are as defined for formula (I).

According to a second advantageous embodiment of the invention, preferred compounds are compounds of formula (IB):

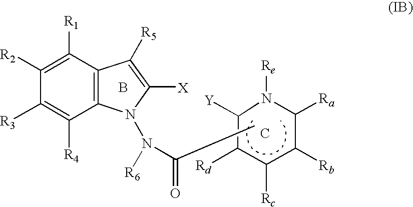

wherein [symbol] X, Y, R₁, R₂, R₃, R₄, R₅, R₆, Rₐ, R_{b}, R_{c}, R_{d} and Rₑ are as defined for formula (I).

According to a third advantageous embodiment of the invention, preferred compounds are compounds of formula (IC):

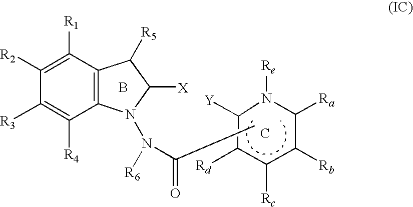

wherein [symbol] X, Y, R₁, R₂, R₃, R₄, R₅, R₆, Rₐ, R_{b}, R_{c}, R_{d} and Rₑ are as defined for formula (I).

According to a fourth advantageous embodiment of the invention, preferred compounds are compounds of formula (ID):

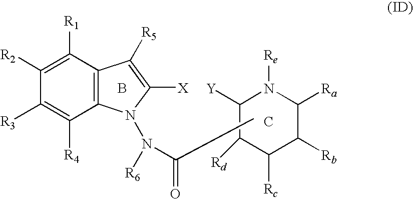

wherein [symbol] X, Y, R₁, R₂, R₃, R₄, R₅, R₆, Rₐ, R_{b}, R_{c}, R_{d} and Rₑ are as defined for formula (I).

According to a fifth advantageous embodiment of the invention, preferred compounds are compounds of formula (IE):

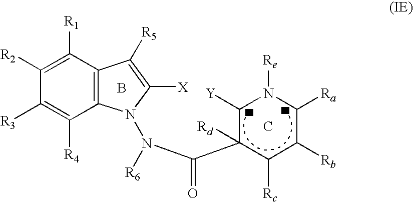

wherein [symbol] X, Y, R₁, R₂, R₃, R₄, R₅, R₆, Rₐ, R_{b}, R_{c}, R_{d} and Rₑ are as defined for formula (I).

According to a sixth advantageous embodiment of the invention, preferred compounds are compounds of formula (IF):

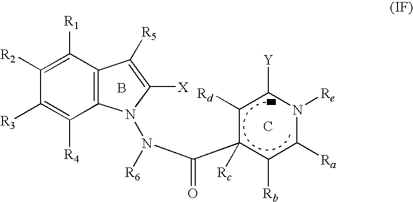

wherein [symbol] X, Y, R₁, R₂, R₃, R₄, R₅, R₆, Rₐ, R_{b}, R_{c}, R_{d} and Rₑ are as defined for formula (I).

According to a seventh advantageous embodiment of the invention, preferred compounds are compounds of formula (IG):

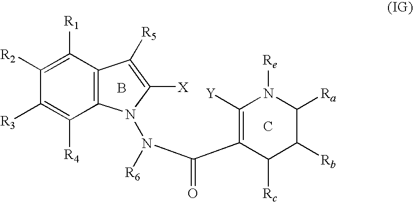

wherein [symbol] X, Y, R₁, R₂, R₃, R₄, R₅, R₆, Rₐ, R_{b}, R_{c}, R_{d} and Rₑ are as defined for formula (I).

According to an eighth advantageous embodiment of the invention, preferred compounds are compounds of formula (IH):

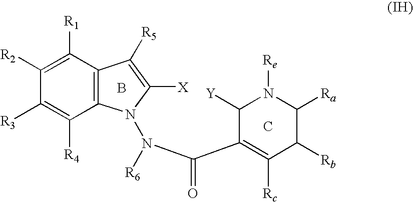

wherein [symbol] X, Y, R₁, R₂, R₃, R₄, R₅, R₆, Rₐ, R_{b}, R_{c}, R_{d} and Rₑ are as defined for formula (I).

According to a ninth advantageous embodiment of the invention, preferred compounds are compounds of formula (IJ):

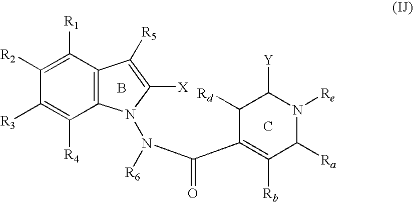

wherein [symbol] X, Y, R₁, R₂, R₃, R₄, R₅, R₆, Rₐ, R_{b}, R_{c}, R_{d} and Rₑ are as defined for formula (I).

Compounds preferred in accordance with the invention are:
N-(1H-indol-1-yl)-1-methyl-1,2,5,6-tetrahydropyridine-3-carboxamide,
N-(2,3-dihydro-1H-indol-1-yl)-1-methyl-1,4,5,6-tetrahydropyridine-3-carboxamide,
N-(5-fluoro-1H-indol-1-yl)-1-methyl-1,2,5,6-tetrahydropyridine-3-carboxamide,
N-(2,3-dihydro-1H-indol-1-yl)-1-methyl-1,4,5,6-tetrahydropyridine-3-carboxamide,
1-[2-(dimethylamino)ethyl]-N-(1H-indol-1-yl)-1,2,5,6-tetrahydropyridine-3-carboxamide,
N-(1H-indol-1-yl)-1-[2-(4-methyl-1-piperazinyl)ethyl]-3-piperidinecarboxamide,
N-(5-chloro-1H-indol-1-yl)-1-(2-hydroxyethyl)-1,4,5,6-tetrahydropyridine-3-carboxamide,
tert-butyl 4-(2-{3-[(1H-indol-1-ylamino)carbonyl]-1-piperidyl}ethyl)piperazine-1-carboxylate,
1-[3-(dimethylammonium)propyl]-3-[(1H-indol-1-ylamino)carbonyl]piperidinium,
N-(1H-indol-1-yl)-1-[3-(1-piperidyl)propyl]-3-piperidinecarboxamide,
N-(1H-indol-1-yl)-1-[3-(4-methyl-1-piperazinyl)propyl]-3-piperidinecarboxamide,
N-(indol-1-yl)-1-(2-piperidin-1-yl-ethyl)-1,2,5,6-tetrahydropyridine-3-carboxamide,
(±)—N-(indol-1-yl)-1-[2-[4-(1-methylpiperidin-4-yl)piperazin-1-yl)]ethyl]piperidine-3-carboxamide,
(±)—N-(indol-1-yl)-1-[3-[4-(2-hydroxyethyl)piperazin-1-yl)]propyl]piperidine-3-carboxamide,
(±)—N-(indol-1-yl)-1-[4-(4-methylpiperazin-1-yl)butyl]piperidine-3-carboxamide,
(±)—N-(indol-1-yl)-1-allylpiperidine-3-carboxamide,
(±)—N-(indol-1-yl)-1-[4-(piperidin-1-yl)but-2-en-1-yl]piperidine-3-carboxamide,
(R or S) (−)—N-(indol-1-yl)-1-[2-(piperidin-1-yl)ethyl]piperidine-3-carboxamide enantiomer 1,
(R or S) (±)—N-(indol-1-yl)-1-[2-(piperidin-1-yl)ethyl]piperidine-3-carboxamide enantiomer 2.

The enantiomers, diastereoisomers, N-oxides, and addition salts with a pharmaceutically acceptable acid or base, of the preferred compounds form an integral part of the invention.

The present invention relates also to a process for the preparation of compounds of formula (I), which process is characterised in that there is used as starting material a compound of formula (II):

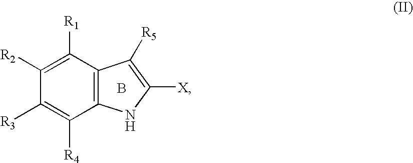

wherein R₁, R₂, R₃, R₄, R₅ and X are as defined for formula (I),
which compound is reacted with diphenylphosphinylhydroxylamine to yield the compound of formula (III):

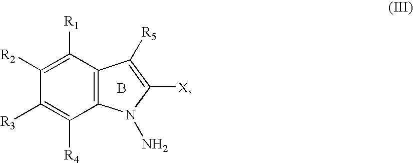

wherein R₁, R₂, R₃, R₄, R₅ and X are as defined hereinbefore,
which compound of formula (III) is condensed with a compound of formula (V):

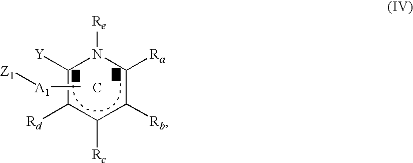

wherein Rₐ, R_{b}, R_{c}, R_{d}, Rₑ and Y are as defined for formula (I), A₁ represents a group of formula —C(=Z)-, —CH₂— or —SO₂— wherein Z is as defined for formula (I) and Z₁ represents a group selected from hydroxy, ethoxy and methoxy,
to yield the compound of formula (I/a), a particular case of the compounds of formula (I):

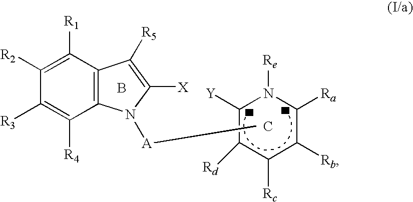

wherein R₁, R₂, R₃, R₄, R₅, Rₐ, R_{b}, R_{c}, R_{d}, Rₑ, X and Y are as defined hereinbefore and A is as defined for formula (I),
the compounds of formula (I/a) forming the entirety of the compounds of the invention, which are purified, if necessary, according to a conventional purification technique, may be separated, when desired, into their different isomers according to a conventional separation technique and are converted, when desired, into their N-oxides and, where appropriate, their addition salts with a pharmaceutically acceptable acid or base.

The compounds of formulae (II) and (IV) are either commercially available or obtained by conventional reactions of organic synthesis well known to the person skilled in the art.

The compound of formula (IV) in the specific case where [symbol] represents a double bond between the carbon atoms carrying substituents R_{c} and R_{d}, of formula (IV/a):

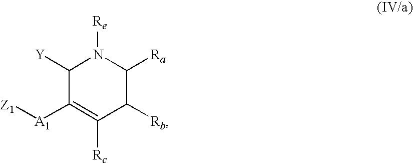

wherein Rₐ, R_{b}, R_{c}, Rₑ, Y, A₁ and Z₁ are as defined hereinbefore,
may especially be obtained starting from the compound of formula (V):

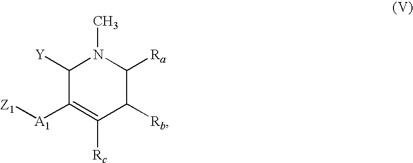

wherein Rₐ, R_{b}, R_{c}, Y, A₁ and Z₁ are as defined hereinbefore,
which is treated with potassium carbonate and chloromethyl chloroformate to yield the compound of formula (VI):

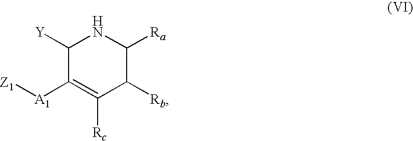

wherein Rₐ, R_{b}, R_{c}, Y, A₁ and Z₁ are as defined hereinbefore,
which compound of formula (VI) is subjected to an alkylation reaction in the presence of a compound of formula (VII):

Rₑ-Hal (VII),

wherein Hal represents a halogen atom and Rₑ is as defined hereinbefore,
to yield the compound of formula (IV/a) as defined hereinbefore.

The compound of formula (IV) in the specific case where [symbol] represents a double bond between the carbon atoms carrying substituents R_{d} and Y, of formula (IV/b):

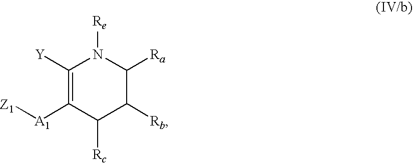

wherein Rₐ, R_{b}, R_{c}, Rₑ, Y, A₁ and Z₁ are as defined hereinbefore,
may especially be obtained starting from the compound of formula (VIII):

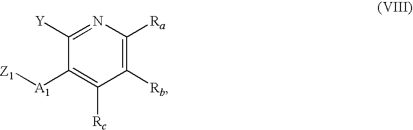

wherein Rₐ, R_{b}, R_{c}, Y, A₁ and Z₁ are as defined hereinbefore,
which is subjected to an alkylation reaction in the presence of a compound of formula (VII) as defined hereinbefore to yield the compound of formula (IX):

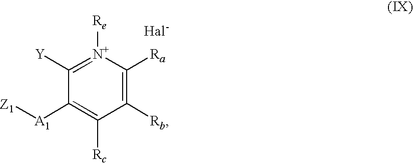

wherein Rₐ, R_{b}, R_{c}, Rₑ, Y, A₁ and Z₁ are as defined hereinbefore and Hal⁻ represents a halide anion,
which compound of formula (IX) is subjected to partial reduction by reaction with triethylamine and PtO₂,
to yield the compound of formula (IV/b) as defined hereinbefore.

The compound of formula (IV) in the specific case where [symbol] represents a single bond, of formula (IV/c):

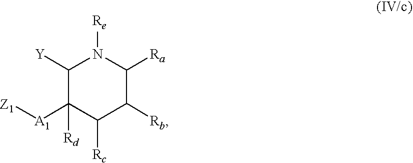

wherein Rₐ, R_{b}, R_{c}, R_{d}, Rₑ, Y, A₁ and Y₁ are as defined hereinbefore,
may especially be obtained starting from the compound of formula (X):

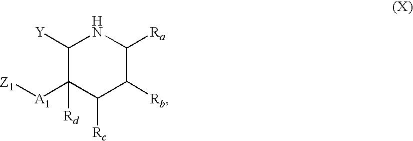

wherein Rₐ, R_{b}, R_{c}, R_{d}, Y, A₁ and Z₁ are as defined hereinbefore,
which is subjected to the action of a compound of formula (VII) as defined hereinbefore to yield the compound of formula (IV/c) as defined hereinbefore.

The compounds of formulae (II), (IV), (V), (VII), (VIII) and (X) are either commercially available or obtained by conventional reactions of organic synthesis well known to the person skilled in the art.

The compounds of formula (I) have valuable pharmacological properties, especially that of being powerful tyrosine hydroxylase (TH) inducers. It is known that tyrosine hydroxylase is a rate-limiting enzyme which controls particularly the synthesis of neurotransmitters in central catecholaminergic and dopaminergic neurons. The rate of synthesis of those neurotransmitters is related especially to the appearance of tonic brain dysfunctions constituting numerous behavioural pathologies in humans, such as anxiety, psychoses, depression, stress etc.

By virtue of their ability to induce tyrosine hydroxylase, the compounds of the invention will accordingly be used therapeutically in the treatment of depression, anxiety, disorders of memory in the course of ageing and/or neurodegenerative diseases, and in the palliative treatment of Parkinson's disease, and for adaptation to stress.

The present invention relates also to pharmaceutical compositions comprising, as active ingredient, at least one compound of formula (I), an enantiomer, diastereoisomer or N-oxide thereof, or an addition salt thereof with a pharmaceutically acceptable acid or base, alone or in combination with one or more pharmaceutically acceptable, inert, non-toxic excipients or carriers.

The pharmaceutical compositions thereby obtained will generally be presented in a dosage form; for example, they may take the form of tablets, dragées, capsules, suppositories, injectable or drinkable solutions and may be administered by the oral, rectal, intramuscular or parenteral route.

Among the pharmaceutical compositions according to the invention there may be mentioned more especially those that are suitable for oral, parenteral (intravenous, intramuscular or subcutaneous), per- or trans-cutaneous, intravaginal, rectal, nasal, perlingual, buccal, ocular or respiratory administration.

The pharmaceutical compositions according to the invention for parenteral injections especially include aqueous and non-aqueous sterile solutions, dispersions, suspensions or emulsions as well as sterile powders for the reconstitution of injectable solutions or dispersions.

The pharmaceutical compositions according to the invention for solid oral administration especially include tablets or dragées, sublingual tablets, sachets, capsules and granules, and for liquid oral, nasal, buccal or ocular administration especially include emulsions, solutions, suspensions, drops, syrups and aerosols.

The pharmaceutical compositions for rectal or vaginal administration are preferably suppositories or ovules, and those for per- or trans-cutaneous administration especially include powders, aerosols, creams, ointments, gels and patches.

The above-mentioned pharmaceutical compositions illustrate the invention but do not limit it in any way.

Among the inert, non-toxic, pharmaceutically acceptable excipients or carriers there may be mentioned, by way of example and without implying any limitation, diluents, solvents, preservatives, wetting agents, emulsifiers, dispersants, binders, swelling agents, disintegrants, retardants, lubricants, absorbency agents, suspension agents, colourants, flavourings etc.

The useful dosage varies according to the age and weight of the patient, the route of administration, the pharmaceutical composition used, the nature and severity of the disorder, and whether any associated treatments are being taken. The dosage ranges from 0.1 mg to 100 mg per day in one or more administrations.

The following Examples illustrate the invention but do not limit it in any way.

The starting materials used are known products or are prepared according to known procedures. The various Preparations yield synthesis intermediates that are useful in preparation of compounds of the invention.

The structures of the compounds described in the Examples and in the Preparations were determined in accordance with the usual spectrometric techniques (infrared, nuclear magnetic resonance, mass spectrometry etc.).

The melting points were determined using a TOTTOLI apparatus (without emergent column correction). When the compound is in the form of a salt, the melting point corresponds to that of the compound in salt form.

### Preparation 1: O-diphenylphosphinylhydroxylamine

To an aqueous solution of 149.44 g of hydroxylamine hydrochloride (340 ml of water) there are added a solution of 72.75 g of sodium hydroxide (290 ml of water) and 960 ml of 1,4-dioxane. The mixture is cooled to −15° C. and then, after 15 minutes, a solution of 180 g of diphenylphosphinyl chloride in 725 ml of dioxane is added all at once with mechanical stirring.

After 5 minutes, 3 litres of water are added all at once. A white precipitate forms which is filtered off and then taken up in 0.25M sodium hydroxide solution at 0° C. The mixture is mechanically stirred at 0° C. for 30 minutes before being filtered again. The precipitate is dried in vacuo (phosphorus pentoxide) to yield 72.5 g of the expected product.

Melting point: 104° C.
Elemental microanalyses:

| | C % | H % | N % |
|---|---|---|---|
| Calculated: | 61.80 | 5.19 | 6.01 |
| Found: | 61.20 | 5.07 | 5.72 |

### Preparation 2: N-aminoindole

To a suspension of 177.72 g of ground potassium hydroxide in 1.3 litres of DMF there are added 21.85 g of indole and then, all at once, 72.5 g of a suspension of the compound of Preparation 1 in 1.3 litres of DMF. The thick mixture is heated at between 60 and 70° C. for 3 hours 30 minutes with mechanical stirring and then, whilst hot, is poured into 3.5 litres of ice-cold water. After cooling, the resulting solution is extracted 3 times using 1.5 litres of ethyl ether. The organic phase is dried over sodium sulphate, filtered and then concentrated under reduced pressure. Chromatography on silica gel (cyclohexane/ether:80/20 and then 50/50) allows 16.5 g of the expected product to be obtained.

Melting point: 35° C.
Elemental microanalyses:

| | C % | H % | N % |
|---|---|---|---|
| Calculated: | 72.70 | 6.10 | 21.20 |
| Found: | 72.68 | 6.14 | 21.17 |

### Preparation 3: 5-Chloro-N-aminoindole

The product is obtained according to the procedure of Preparation 2, using 5-chloroindole instead of indole.

Melting point: 46° C.
Elemental microanalyses:

| | C % | H % | N % |
|---|---|---|---|
| Calculated: | 57.61 | 4.23 | 16.81 |
| Found: | 57.51 | 4.41 | 16.68 |

### Preparation 4: 3-Methyl-N-aminoindole

The product is obtained according to the procedure of Preparation 2, using 3-methylindole instead of indole.

### Preparation 5: 2,3-Dimethyl-N-aminoindole

The product is obtained according to the procedure of Preparation 2, using 2,3-dimethyl-indole instead of indole.

### Preparation 6: N-aminoindoline

### Step A: 1-Nitrosoindoline

To a solution, cooled to 0° C., of 4 g of indoline in 100 ml of 50% aqueous acetic acid there is added, dropwise, a solution of 2.32 g of NaNO₂ in 50 ml of water; the solution is then stirred for 30 minutes at 0° C. The suspension is then made alkaline by adding 190 ml of 20% aqueous sodium hydroxide solution and is then extracted three times with ethyl acetate. The combined organic phases are dried over sodium sulphate, filtered and then evaporated. 4.92 g of the expected product are obtained.

Melting point: 68° C.
Elemental microanalyses:

| | C % | H % | N % |
|---|---|---|---|
| Calculated: | 64.85 | 5.44 | 18.91 |
| Found: | 64.67 | 5.56 | 18.69 |

### Step B: N-aminoindoline

4 g of the compound of Step A above are dissolved in 50 ml of a mixture of diethyl ether/dichloromethane 85/15 and then poured dropwise into a solution of 1.4 g of LiAlH₄ 1M/Et₂O (36 ml) at 0° C. After adding for 30 minutes, the reaction mixture is stirred for 3 hours at ambient temperature, and then 1.5 ml of water, 4.5 ml of 15% sodium hydroxide solution and then 1.5 ml of water are added slowly. The aluminium salts are filtered off over Celite (eluting with dichloromethane) and the solvents are then evaporated off under reduced pressure. Chromatography on silica gel (cyclohexane/diethyl ether: 7/3) allows 2.9 g of the expected product to be isolated in the form of an orange oil.

Infrared (ν_{cm-1}):

3335 (νN—H); 3046; 3025 (ν═C—H); 2954; 2844 (νC—H); 1605 (νC═C); 1478 (

### δC—H).

### Preparation 7: Methyl 1-methyl-1,4,5,6-tetrahydropyridine-3-carboxylate

### Step A: 3-(Methoxycarbonyl)-1-methylpyridinium Iodide

10 g of methyl nicotinate are dissolved in 5.2 ml of methyl iodide and then the reaction mixture is heated at 60° C. for 24 hours protected from light. The residue is purified by flash chromatography on silica gel (CH₂Cl₂/CH₃OH: 9/1 and then 85/15), allowing 13.46 g of the expected product to be isolated.

### Step B: Methyl 1-methyl-1,4,5,6-tetrahydropyridine-3-carboxylate

4 g of the compound of Step A above, diluted with 100 ml of absolute methanol, are hydrogenated at atmospheric pressure for 20 hours at 20° C. in the presence of 4 ml of triethylamine and 800 mg of 10% Pd/C. After filtering off the catalyst over Celite and washing (twice with methanol), the filtrate is evaporated in vacuo and yields a yellow residue. The residue is carefully washed three times with distilled diethyl ether and then, after evaporating off the solvent in vacuo, 2.15 g of a yellow oil are isolated.

### Preparation 8: 2-Methyl-1-indolinamine

The product is obtained according to the procedure of Preparation 6, using 2-methyl-indoline instead of indoline.

### Preparation 9: 5-Fluoro-N-aminoindole

The product is obtained according to the procedure of Preparation 2, using 5-fluoroindole instead of indole.

### Preparation 10: 5-Methoxy-N-aminoindole

The product is obtained according to the procedure of Preparation 2, using 5-methoxy-indole instead of indole.

### Preparation 11: Ethyl 1-allylpiperidine-3-carboxylate

2 g of ethyl piperidine-3-carboxylate are added to 1.6 g of allyl bromide, and then 13 ml of benzene are added. 1.08 g of sodium carbonate are added and the reaction mixture is stirred overnight at the reflux of benzene. The reaction mixture is filtered and then evaporated under reduced pressure. Purification by distillation using a glass oven allows 1.53 g of the expected product to be isolated.

T_{boiling}(8×10⁻² bar) 80-85° C.

INFRARED (ν_{cm-1})

3078 (ν═C—H); 2980; 2941; 2866 (νC—H); 2791 (νN—CH₂); 1733 (νC═O); 1644 (νC═C); 1468; 1453 (δC—H); 1368 (νC—N); 1223; 1182 (νC—O).

### Preparation 12: Ethyl 1-methylpiperidine-3-carboxylate

3 g of ethyl piperidine-3-carboxylate, 4 ml of aqueous formaldehyde, 300 mg of 10% Pd/C and 4 ml of glacial acetic acid are placed under an atmosphere of hydrogen (1 atm.) at 20° C. for 17 hours. After filtering off the catalyst over Celite and washing with 50 ml of ethanol, the solution is evaporated under reduced pressure and yields an oily residue. The residue is diluted with a mixture of toluene/water (1/1) and the pH is adjusted to 9 by adding 20% K₂CO₃. After separation of the two phases, the aqueous phase is extracted twice with toluene. The organic phases are washed with water, dried over Na₂SO₄ and yield a lightly coloured oil. Distillation under reduced pressure yields 2.4 g of the expected product.

Boiling point: 105-110° C. (P=20 mmHg)

### Preparation 13: Ethyl 1-propylpiperidine-3-carboxylate

2 g of ethyl piperidine-3-carboxylate are added to 1.6 g of 1-bromopropane, and then 13 ml of benzene are added. 1.08 g of sodium carbonate are added and the reaction mixture is stirred overnight at the reflux of benzene. The reaction mixture is filtered and then evaporated under reduced pressure. Purification by distillation using a glass oven allows 1.53 g of the expected product to be isolated.

### Preparation 14: Methyl 1-(2-hydroxyethyl)-1,2,5,6-tetrahydropyridine-3-carboxylate

### Step A: Methyl 1,2,5,6-tetrahydropyridine-3-carboxylate

7 g of arecoline hydrobromide are dissolved in 20 ml of water, and then the solution is made alkaline by adding 5.13 g of potassium carbonate followed by saturation with NaCl. The aqueous phase is extracted three times with ether. The combined organic phases are dried over sodium sulphate, filtered and then evaporated until a weight of 4.7 g of a colourless oil is obtained. The oil is diluted with 33 ml of anhydrous toluene, and then 3.92 ml of 1-chloroethyl chloroformate are added. A precipitate forms and the mixture is heated overnight at the reflux of toluene. The precipitate is filtered off and then the organic phase is washed with 0.1M hydrochloric acid solution; the aqueous phase is extracted once with ether. The combined organic phases are dried over sodium sulphate, filtered and then evaporated under reduced pressure. The residue is taken up in 25 ml of methanol and is then refluxed for 2 hours. The methanol is evaporated off under reduced pressure, and 3.8 g of methyl 1,2,5,6-tetrahydropyridine-3-carboxylate hydrochloride are obtained. The expected product is obtained by dissolving the methyl 1,2,5,6-tetrahydropyridine-3-carboxylate hydrochloride in water and then making alkaline by adding potassium carbonate until a pH of 10 is achieved. Saturation with sodium chloride is carried out and then the aqueous phase is extracted three times with ether; the combined organic phases are dried over sodium sulphate, filtered and then evaporated until 3 g of the expected product are obtained.

### Step B: Methyl 1-(2-hydroxyethyl)-1,2,5,6-tetrahydropyridine-3-carboxylate

To a solution of 1.77 g of the compound of Step A above in 17 ml of anhydrous 1,4-dioxane there are added 5.22 g of potassium carbonate, 24 mg of sodium iodide and then 900 μl of bromoethanol. The reaction mixture is refluxed overnight with stirring and is then concentrated under reduced pressure. The residue is taken up in water, and the aqueous phase is extracted twice with ethyl acetate. The combined organic phases are dried over sodium sulphate, filtered and then evaporated, allowing 1.75 g of the expected product to be isolated.

Infrared (ν_{cm-1}):

3398 (νO—H); 2950; 2815 (νC—H); 1710 (νC═O); 1656 (νC═C).

### Preparation 15: Methyl 1-(2{[tert-butyl(dimethyl)silyl]oxy}ethyl)-1,2,5,6-tetrahydropyridine-3-carboxylate

To a solution of 1.5 g of the compound of Preparation 14 in 15 ml of pyridine there are added 2.08 g of tert-butyldimethylsilyl chloride. The solution is stirred overnight at ambient temperature. The solid obtained is dissolved in dichloromethane and then washed with a pH 11 solution of sodium carbonate; the aqueous phase is extracted with dichloromethane. The combined organic phases are dried over sodium sulphate, filtered and then evaporated under reduced pressure. Flash chromatography on silica gel (cyclohexane/AcOEt:3/1 and then 2/1) allows 1.9 g of the expected product to be isolated.

Infrared (ν_{cm-1})

2951; 2929; 2856 (νC—H); 1716 (νC═O); 1658 (νC═C).

### Preparation 16: Methyl 1-(2-dimethylaminoethyl)-1,2,5,6-tetrahydropyridine-3-carboxylate

To a solution of 1.94 g of the compound of Step A of Preparation 14 in 27 ml of anhydrous 1,4-dioxane there are added 2.03 g of sodium iodide, 4.3 g of sodium carbonate and 1.45 g of 2-dimethylaminoethyl chloride. The reaction mixture is stirred overnight at the reflux of dioxane, and the reaction mixture is then filtered and rinsed with dichloromethane. The filtrate is evaporated under reduced pressure. The residue is taken up in dichloromethane, washed with a pH 10 solution of sodium carbonate, dried over Na₂SO₄, filtered and then evaporated under reduced pressure. Purification using a glass oven allows 846 mg of the expected product to be isolated.

Infrared (ν_{cm-1})

2948; 2816 (νC—H); 2765 (νN—CH₃); 1712 (νC═O); 1657 (νC═C).

### Preparation 17: Methyl 142-hydroxyethyl)-1,4,5,6-tetrahydropyridine-3-carboxylate

### Step A: 1-(2-Hydroxyethyl)-3-(methoxycarbonyl)pyridinium bromide

10 g of methyl nicotinate are dissolved in 5.2 ml of 2-bromoethanol and then the reaction mixture is heated at 60° C. for 24 hours protected from light. The residue is purified by flash chromatography on silica gel (CH₂Cl₂/CH₃OH: 9/1 and then 85/15), allowing 13.46 g of the expected product to be isolated.

Melting point: 90° C.
Elemental microanalyses:

| | C % | H % | N % |
|---|---|---|---|
| Calculated: | 41.24 | 4.61 | 5.34 |
| Calculated + H₂O: | 38.59 | 5.04 | 5.00 |
| Found: | 37.99 | 5.01 | 4.83 |

### Step B: Methyl 1-(2-hydroxyethyl)-1,4,5,6-tetrahydropyridine-3-carboxylate

To a solution of 4 g of the compound of Step A above in 100 ml of rectified MeOH there are added 2.73 ml of triethylamine and 400 mg of 10% Pd/C. The mixture is degassed twice in vacuo and placed under hydrogen. The reaction mixture is stirred for 18 hours and is then filtered over Celite (eluting with CH₂Cl₂). The solution is evaporated under reduced pressure. The residue is taken up in 100 ml of diethyl ether and water; the aqueous phase is extracted twice with ether, and 2.41 g of the expected product are obtained.

Melting point: 38° C.
Elemental microanalyses:

| | C % | H % | N % |
|---|---|---|---|
| Calculated: | 58.36 | 8.16 | 7.56 |
| Found: | 57.65 | 8.41 | 7.38 |

Preparation 18: Methyl 1-[(2-dimethylamino)ethyl]-1,4,5,6-tetrahydropyridine 3-carboxylate

To a solution of 2 g of the compound of Preparation 17 in 20 ml of anhydrous dichloromethane there are added 2 ml of triethylamine and then 1.1 ml of mesyl chloride at 0° C. The reaction mixture is stirred for 30 minutes at ambient temperature and then 20 ml of water are added; the organic phase is diluted with dichloromethane. The aqueous phase is extracted twice with dichloromethane. The combined organic phases are dried over Na₂SO₄, filtered and then evaporated under reduced pressure. The residue is taken up in 20 ml of acetonitrile, and then 12.5 ml of 2M dimethylamine in MeOH are added. The reaction mixture is stirred for 15 hours at ambient temperature and then for 2 hours at 50° C. The reaction mixture is concentrated under reduced pressure, taken up in dichloromethane and washed with a pH 11 solution of K₂CO₃ and then with saturated NaCl solution. The organic phase is dried over Na₂SO₄, filtered and then evaporated under reduced pressure, allowing 1.42 g of the expected product to be isolated.

Infrared (V_{cm-1})

2961; 2853 (νC—H); 2771 (νN—CH₃); 1678 (νC═O); 1617 (νC═C).

### Preparation 19: Ethyl 1-[2-(dimethylamino)ethyl]piperidine-3-carboxylate

3.51 g of 2-dimethylaminoethyl chloride are dissolved in 50 ml of anhydrous 1,4-dioxane and there are then added 9.58 g of sodium carbonate and 4.52 g of sodium iodide, followed by 5.16 g of ethyl piperidine-3-carboxylate. The reaction mixture is refluxed for 20 hours and then the dioxane is evaporated off under reduced pressure. The residue is taken up in ethyl acetate and washed with water and then with saturated sodium chloride solution. The organic phase is dried over sodium sulphate and then evaporated under reduced pressure. Purification by distillation using a glass oven allows 3.41 g of the expected product to be isolated.

Boiling point=90° C. (5×10⁻¹ bar)

Infrared (ν_{cm-1}):

2941; 2857; 2815 (νC—H); 2766 (νN—CH₃); 1730 (νC═O); 1464 (δC—H).

### Preparation 20: Ethyl 1-[2-(4-morpholinyl)ethyl]piperidine-3-carboxylate

2.0 g of the compound of Preparation 36 and 2.3 ml of morpholine are dissolved in 15 ml of 70% ethanol. The reaction mixture is stirred at ambient temperature for 96 hours. The solution is then concentrated and subsequently taken up in 30 ml of dichloromethane. The organic phase is then washed several times with water, dried over Na₂SO₄, filtered and then concentrated. Chromatography on silica gel (CH₂Cl₂/MeOH: 9/1) allows 2.3 g of the expected product to be isolated.

Infrared (ν_{cm-1})

2943, 2854, 2810 (νC—H), 1729 (νC═O ester), 1449 (δC—H), 1372 (νC—N), 1117 (νC—O—C).

### Preparation 21: Ethyl 1-[2-(4-piperidyl)ethyl]piperidine-3-carboxylate

The product is obtained according to the procedure of Preparation 20, using piperidine instead of morpholine.

Infrared (ν_{cm-1}):

2939, 2859 (νC—H), 1726 (νC═O ester), 1453 (δC—H), 1372 (νC—N), 1183, 1154 (νC—O).

### Preparation 22: Ethyl 1-[2-(4-methyl-1-piperazinyl)ethyl]piperidine-3-carboxylate

The product is obtained according to the procedure of Preparation 20, using N-methylpiperazine instead of morpholine.

Infrared (ν_{cm-1}):

2939, 2799 (νC—H), 1731 (νC═O ester), 1452 (δC—H), 1373 (νC—N).

### Preparation 23: Ethyl 1-{2-[4-(2-hydroxyethyl)-1-piperazinyl]ethyl}piperidine-3-carboxylate

The product is obtained according to the procedure of Preparation 20, using (2-hydroxy-ethyl)piperazine instead of morpholine.

Infrared (ν_{cm-1}):

3235 (νO—H), 2940, 2810 (νC—H), 1730 (νC═O ester), 1451 (δC—H), 1371 (νC—N), 1222 (νC—O).

### Preparation 24: Ethyl 1-[2-(1-pyrrolidinyl)ethyl]piperidine-3-carboxylate

The product is obtained according to the procedure of Preparation 20, using pyrrolidine instead of morpholine.

Infrared (ν_{cm-1})

2942, 2782 (νC—H), 1732 (νC═O ester), 1452 (δC—H), 1371 (νC—N), 1226 (νC—O).

### Preparation 25: Ethyl 1-[2-(1-azepanyl)ethyl]piperidine-3-carboxylate

The product is obtained according to the procedure of Preparation 20, using hexamethyleneimine instead of morpholine.

Infrared (ν_{cm-1})

2925, 2853, 2810 (νC—H), 1733 (νC═O ester), 1450 (δC—H), 1370 (νC—N), 1225 (νC—O).

### Preparation 26: Ethyl 1-[2-(4-phenyl-1-piperazinyl)ethyl]piperidine-3-carboxylate

The product is obtained according to the procedure of Preparation 20, using 4-phenyl-piperazine instead of morpholine.

Infrared (ν_{cm-1})

2942, 2817 (νC—H), 1731 (νC═O ester), 1600 ((νC═C), 1502, 1451 (νC—H).

### Preparation 27:1-[2-(1-Piperidyl)ethyl]piperidine-3-carbaldehyde

0.98 g of the compound of Preparation 18 is dissolved in 30 ml of anhydrous THF and then the solution is cooled to −80° C. 2.5 ml of a solution of DIBAL-H (1.5M in toluene) are then slowly added by syringe. The solution is then stirred at −80° C. and then, after 1.5 hours, a further 2.5 ml of the DIBAL-H solution are added to the mixture. After reacting for 3 hours at −80° C., 15 ml of water are added and the temperature is then allowed to come back up to ambient temperature. The solution is then extracted with two 50 ml quantities of CH₂Cl₂ and then washed with saturated NaCl solution. After drying over Na₂SO₄ and concentrating, the expected product is obtained and is used without further purification in further steps.

### Preparation 28: 5-Chloro-N-aminoindoline

### Step A: 1-Acetylindoline

23 ml of acetic anhydride are added dropwise to 5 g of indoline, whilst maintaining the temperature of the mixture at 0°. The mixture is refluxed for 4 hours, with stirring, and is then evaporated under reduced pressure. Flash chromatography on silica gel (cyclohexane/AcOEt: 6/4 and then pure AcOEt) allows 6.4 g of the expected product to be isolated.

### Step B: 5-Chloroindoline

3.84 g of SO₂Cl₂ are added dropwise at 0° C. to a solution of 6.4 g of the compound of Step A above in 170 ml of carbon tetrachloride. A white precipitate forms, which is stirred at ambient temperature for one hour. The suspension is diluted with dichloromethane and with water. The organic phase is extracted and then washed with 20% aqueous sodium hydroxide solution. The organic phase is dried over sodium sulphate, filtered and then evaporated under reduced pressure. The residue is taken up in 110 ml of absolute ethanol and refluxed until dissolution is complete. 49 ml of 37% HCl are added and the solution is then stirred under reflux for 3 hours 30 minutes. The ethanol is evaporated off under reduced pressure, and the aqueous phase is then diluted with 50 ml of water, extracted with ether and then made alkaline using 20% aqueous sodium hydroxide solution. The aqueous phase is extracted three times with ether. The combined organic phases are dried over sodium sulphate, filtered and then evaporated under reduced pressure. Flash chromatography on silica gel (cyclohexane/AcOEt:7/3) allows 3.53 g of the expected product to be isolated.

### Step C: 5-Chloro-1-nitrosoindoline

To a solution, cooled to 0° C., of 3.53 g of the compound of Step B above in 70 ml of 50% aqueous acetic acid there is added, dropwise, a solution of 1.59 g of NaNO₂ in 35 ml of water; the solution is then stirred for 30 minutes at 0° C. The mixture is then made alkaline by adding 130 ml of 20% aqueous sodium hydroxide solution and is then extracted three times with ethyl acetate. The combined organic phases are dried over sodium sulphate, filtered and then evaporated under reduced pressure, allowing 4.15 g of the expected product to be isolated.

Melting point: 89° C.
Elemental microanalyses:

| | C % | H % | N % |
|---|---|---|---|
| Calculated: | 52.62 | 3.86 | 15.34 |
| Found: | 53.86 | 4.41 | 15.45 |

### Step D: 5-Chloro-N-aminoindoline

A suspension of 570 mg of LiAlH₄ in 15 ml of anhydrous ether is stirred at the reflux of ether for 45 minutes and is then stirred overnight at ambient temperature. 2 g of the compound of Step C above are dissolved in 6 ml of anhydrous THF and then diluted with 14 ml of anhydrous ether. The solution thereby obtained is added dropwise to the solution of LiAlH₄ at 0° C. The solution is stirred for three hours at 0° C., and then 0.5 ml of water, 1.5 ml of 15% aqueous sodium hydroxide solution and 0.5 ml of water are gently added under nitrogen. The aluminium salts are filtered off over Celite (eluting with dichloromethane). The solution is evaporated under reduced pressure. Flash chromatography on silica gel (cyclohexane/AcOEt:85/15 and then 8/2 to 7/3) allows 1.7 g of the expected product to be isolated.

Melting point: 30° C.
Elemental microanalyses:

| | C % | H % | N % |
|---|---|---|---|
| Calculated: | 56.98 | 5.38 | 16.61 |
| Found: | 58.13 | 5.61 | 16.44 |

### Preparation 29: Methyl 1-(2-{[tert-butyl(dimethyl)silyl]oxy}ethyl)-1,4,5,6-tetrahydro-3-pyridinecarboxylate

The product is obtained according to the procedure of Preparation 15, using the compound of Preparation 17 instead of the compound of Preparation 14.

### Preparation 30: Ethyl 1-(2-hydroxyethyl)piperidine-3-carboxylate

2 g of ethyl piperidine-3-carboxylate are added to 1.6 g of 2-bromoethanol and then diluted with 13 ml of benzene. 1.08 g of sodium carbonate are added and the reaction mixture is stirred overnight at the reflux of benzene. The reaction mixture is filtered and then evaporated under reduced pressure. Purification by distillation using a glass oven allows 1.53 g of the expected product to be isolated.

Boiling point: 100° C. (3×10⁻² bar)

Infrared (ν_{cm-1})

3412 (νO—H); 2941; 2808 (νC—H); 1730 (νC═O); 1468 (δC—H).

### Preparation 31: Ethyl 1-benzylpiperidine-3-carboxylate

The product is obtained according to the procedure of Preparation 30, using benzyl bromide instead of 2-bromoethanol.

### Preparation 32: Methyl 4-(4-fluorophenyl)-1-methylpiperidine-3-carboxylate

2.5 g of arecoline are dissolved in 20 ml of anhydrous diethyl ether and 25 ml of anhydrous dichloromethane. The solution is cooled to −30° C. and 32.2 ml of 1M (4-fluorophenyl)-magnesium bromide solution are added dropwise. The mixture is stirred for 3 hours at a temperature from −30° C. to −35° C. and is then cooled to −78° C. for 30 minutes. 10 ml of a 1/1 mixture of trifluoroacetic acid and ether are then added dropwise; the mixture is then brought to 0° C. and 15 ml of 1M hydrochloric acid solution are added, followed by 28% ammonium hydroxide solution to bring to pH 12. The organic phase is collected and the aqueous phase is extracted three times with ethyl acetate. The combined organic phases are dried over sodium sulphate, filtered and then evaporated under reduced pressure. Flash chromatography on silica gel (cyclohexane/triethylamine:3/1) allows the expected product to be isolated.

### Preparation 33: tert-Butyl 4-{2-[3-(ethoxycarbonyl)-1-piperidyl]ethyl}-piperazine-1-carboxylate

The expected product is obtained according to the procedure of Preparation 20, using tert-butyl piperazine-1-carboxylate instead of morpholine.

Infrared (ν_{cm-1})

2938, 2811 (νC—H); 1731 (νC═O ester); 1698 (νC═ONBOc); 1455, 1421, 1373 (νC—N)

### Preparation 34: Ethyl 1-[3-(dimethylamino)propyl]piperidine-3-carboxylate

1.5 g of the compound of Preparation 24 and 4.8 ml of dimethylamine are dissolved in 10 ml of 70% ethanol. The reaction mixture is stirred at ambient temperature for 48 hours. A further 4.8 ml of dimethylamine are added and the reaction mixture is then heated at 50° C. for 24 hours. The solution is then concentrated using a rotary evaporator and subsequently taken up in 50 ml of dichloromethane. The organic phase is then washed several times with water, dried over Na₂SO₄, filtered and then concentrated. Chromatography on silica gel (CH₂Cl₂/MeOH: 9/1) allows 0.81 g of the expected product to be isolated.

Infrared (ν_{cm-1})

2942, 2813, 2761 (νC—H); 1730 (νC═O ester); 1466 (δC═H); 1373 (νC—N); 1235, 1179 (νC═O), 1104

### Preparation 35: Ethyl 1-(3-hydroxypropyl)piperidine-3-carboxylate

The product is obtained according to the procedure of Preparation 30, using 3-bromopropanol instead of 2-bromoethanol.

Boiling point: 125° C. (3×10⁻² bar)

Infrared (ν_{cm-1})

3388 (νO—H); 2941; 2861; 2811 (νC—H) 1729 (νC═O); 1470 (δC—H).

### Preparation 36: Ethyl 1-(2-chloroethyl)piperidine-3-carboxylate

10.0 g of ethyl 3-piperidine-3-carboxylate are dissolved in 80 ml of acetone, and then 11 ml of 1-bromo-2-chloroethane and 14 g of potassium carbonate are added at ambient temperature. After stirring for 40 hours, the solvent is evaporated off and then 50 ml of water and 100 ml of diethyl ether are added. The organic phase is separated off, washed with water, dried over Na₂SO₄ and then concentrated. Chromatography on silica gel (petroleum ether/ethyl ether: 6/4) allows 9.24 g of the expected product to be isolated.

Infrared (ν_{cm-1})

2943, 2808 (νC—H), 1729 (νC═O ester), 1468, 1450 (δC—H), 1370 (νC—N), 1209, 1179 (νC—O).

### Preparation 37: Ethyl 1-(3-chloropropyl)piperidine-3-carboxylate

The product is obtained according to the procedure of Preparation 36, using 1-bromo-3-chloropropane instead of 1-bromo-2-chloropropane.

Infrared (ν_{cm-1})

2945, 2808 (νC—H), 1730 (νC═O ester), 1469, 1446 (νC—H), 1371 (νC—N), 1179, 1153 (νC—O).

### Preparation 38: Ethyl 1-[3-(1-piperidyl)propyl]piperidine-3-carboxylate

The product is obtained according to the procedure of Preparation 20, using piperidine instead of morpholine and the compound of Preparation 37 instead of the compound of Preparation 36.

Infrared (ν_{cm-1})

2933, 2854, 2802, 2761 (νC—H), 1731 (νC═O ester), 1469, 1463 (δC—H), 1373 (νC—N), 1271, 1178 (νC—O).

### Preparation 39: Ethyl 1-[3-(4-methyl-1-piperazinyl)propyl]piperidine-3-carboxylate

The product is obtained according to the procedure of Preparation 20, using N-methyl-piperazine instead of morpholine and the compound of Preparation 37 instead of the compound of Preparation 36.

Infrared (ν_{cm-1})

2939, 2794 (νC—H), 1730 (νC═O ester), 1448 (δC—H), 1372 (νC—N), 1283, 1150 (νC—O).

### Preparation 40: Ethyl 1-(2-{[tert-butyl(dimethyl)silyl]oxy}ethyl)piperidine-3-carboxylate

1.276 g of tert-butyldimethylsilyl chloride are added to a solution of 1 g of the compound of Preparation 30 in 16 ml of pyridine. The reaction mixture is stirred overnight at ambient temperature, and then the pyridine is evaporated off under reduced pressure (co-evaporation with toluene). The residue is taken up in dichloromethane, washed twice with water and then with saturated NaCl solution. The organic phase is dried over sodium sulphate, filtered and then evaporated. Flash chromatography on silica gel (cyclohexane/AcOEt:3/1) allows 1.1 g of the expected product to be isolated.

Infrared (ν_{cm-1})

2930; 2857 (νC—H); 1733 (νC═O); 1470 (δC—H).

### Preparation 41: Ethyl 1-(3-{[tert-butyl(dimethyl)silyl]oxy}propyl)piperidine-3-carboxylate

The product is obtained according to the procedure of Preparation 40, using the compound of Preparation 35 instead of the compound of Preparation 30.

Infrared (ν_{cm-1})

2951; 2930; 2857 (νC—H); 1734 (νC═O); 1471 (δC—H).

### Preparation 42: Methyl 1-(2-chloroethyl)-1,2,5,6-tetrahydropyridine-3-carboxylate

### Step A: Guvacine hydrochloride

7 g of arecoline hydrobromide are dissolved in 20 ml of water, the solution is made alkaline by adding 5.13 g of potassium carbonate and is then saturated with NaCl. The aqueous phase is extracted three times with diethyl ether. The combined organic phases are dried over sodium sulphate, filtered and then evaporated until a weight of 4.7 g of a colourless oil is obtained. The oil is taken up in 20 ml of toluene; the solution is turbid. After adding sodium sulphate and filtering, the insoluble material is washed with 13 ml of toluene. 3.92 ml of 1-chloroethyl chloroformate are added to the organic solution. A precipitate forms and the reaction mixture is heated for 12 hours at the reflux of toluene. The precipitate is filtered off and then the organic phase is washed with 0.1M aqueous hydrochloric acid solution; the aqueous phase is extracted once with diethyl ether. The combined organic phases are dried over sodium sulphate, filtered and then evaporated under reduced pressure. The residue is taken up in 25 ml of methanol and is then heated at reflux for 2 hours. The methanol is evaporated off under reduced pressure, and 3.8 g of the expected product are obtained in a yield of 73%. Guvacine base is obtained by dissolving the hydrochloride in water; the aqueous phase is made alkaline by adding potassium carbonate until a pH of 10 is achieved, and it is saturated with NaCl. The aqueous phase is extracted three times with diethyl ether and the combined organic phases are dried over sodium sulphate, filtered and then evaporated until 3 g of a colourless oil are obtained.

Elemental microanalyses:

| | C % | H % | N % |
|---|---|---|---|
| Calculated: | 47.33 | 6.81 | 7.89 |
| Found: | 47.38 | 6.93 | 7.83 |

### Step B: Methyl 1-(2-chloroethyl)-1,2,5,6-tetrahydropyridine-3-carboxylate

To a suspension of 530 mg of the compound of Step A above in 12 ml of acetone there are added 2.53 ml of triethylamine and 1.1 ml of 1-bromo-2-chloroethane. The mixture is stirred at ambient temperature for 18 hours and then heated at reflux for 8 hours. It is evaporated under reduced pressure, taken up in dichloromethane and washed with aqueous potassium carbonate solution. The aqueous phase is extracted with dichloromethane. The combined organic phases are dried over sodium sulphate, filtered and then evaporated under reduced pressure. The residue is purified by flash chromatography over silica gel (cyclohexane/AcOEt:7/3) allowing 430 mg of the expected compound to be obtained.

Elemental microanalyses:

| | C % | H % | N % |
|---|---|---|---|
| Calculated: | 53.08 | 6.93 | 6.88 |
| Found: | 53.74 | 7.22 | 6.72 |

INFRARED (ν_{cm-1}): 2951; 2917; 2811 (νC—H); 2767 (νN—CH₂); 1709 (νC═O); 1657 (νC═C); 1462; 1436 (δC—H); 1375 (νC—N); 1261 (νC—O).

### Preparation 43: Methyl 1-[2-(4-methylpiperazin-1-yl)ethyl]-1,2,5,6-tetrahydropyridine-3-carboxylate

To a solution of 320 mg of the compound of Preparation 42 in 5 ml of 70% aqueous ethanol there are added 540 μl of 1-methylpiperazine. The solution is stirred at ambient temperature for 72 hours and is then evaporated under reduced pressure. The residue is taken up in dichloromethane and washed twice with aqueous sodium carbonate solution. The organic phase is dried over sodium sulphate, filtered and evaporated under reduced pressure to obtain 320 mg of the expected compound.

Infrared (ν_{cm-1})

2938 (νC—H); 2793 (νN—CH₃); 1712 (νC═O); 1657 (νC═C); 1438 (δC—H); 1373 (νC—N); 1262 (νC—O).

### Preparation 44: 5-Methyl-N-aminoindole

The product is obtained according to the procedure of Preparation 2 using 5-methylindole instead of indole.

### Preparation 45: Methyl 1-[2-[4-[2-(tert-butyldimethylsilanoxy)ethyl]piperazin-1-yl]ethyl]-1,2,5,6-tetrahydropyridine-3-carboxylate

### Step A: Methyl 1-[2-[4-(2-hydroxyethyl)piperazin-1-yl]ethyl]-1,2,5,6-tetrahydropyridine-3-carboxylate

The product is obtained according to the procedure of Preparation 43 using 4-(2-hydroxyethyl)piperazine instead of 1-methylpiperazine.

Infrared (ν_{cm-1})

3350 (νO—H); 2946 (νC—H) 2812 (νN-—(CH₂); 1710 (νC═O); 1656 (νC═C); 1437 (δC—H); 1351 (νC—N); 1262 (νC—0).

### Step B: Methyl 1-[2-[4-[2-(tert-butyldimethylsilanoxy)ethyl]piperazin-1-yl]ethyl]-1,2,5,6-tetrahydropyridine-3-carboxylate

To a solution of 340 mg of the compound of Step A above in 5 ml of pyridine there are added 260 mg of tributyldimethylsilyl chloride. The solution is stirred at ambient temperature for 16 hours and then the pyridine is evaporated off under reduced pressure. After dissolving the residue in dichloromethane, the organic phase is extracted with aqueous potassium carbonate solution. The aqueous phase is extracted with dichloromethane. The combined organic phases are dried over sodium sulphate, filtered and evaporated under reduced pressure. Flash chromatography over silica gel (CH₂Cl₂/CH₃OH: 9/1, then 85/15) allows 360 mg of the expected product to be obtained.

### Preparation 46: Methyl 1-[2-(piperidin-1-yl)ethyl]-1,2,5,6-tetrahydropyridine-3-carboxylate

The product is obtained according to the procedure of Preparation 43 using piperidine instead of 1-methylpiperazine.

Infrared (ν_{cm-1})

2955; 2932; 2872 (νC—H); 1736 (νC═O); 1661 (νC═C); 1434 (δC—H); 1368; 1341 (νC—N); 1236; 1194 (νC—O).

### Preparation 47: 1,2-Bis[3-(ethoxycarbonyl)-1,2,5,6-tetrahydropyridin-1-yl]-ethane

To a solution of 900 mg of the compound of Step A of Preparation 42 in 10 ml of methanol there is added 0.32 ml of 2-bromochloroethane, followed by 1.6 ml of triethylamine. The mixture is heated at reflux for 20 hours and is evaporated under reduced pressure. The residue is dissolved in dichloromethane and extracted with saturated aqueous potassium carbonate solution. The aqueous phase is extracted with dichloromethane. The combined organic phases are dried over sodium sulphate, filtered and evaporated under reduced pressure. Flash chromatography over silica gel (AcOEt, then AcOEt/MeOH:95/5 to 90/10) allows 500 mg of the expected product to be obtained.

Melting point: 77° C.
Elemental microanalyses:

| | C % | H % | N % |
|---|---|---|---|
| Calculated: | 61.13 | 7.91 | 8.91 |
| Found: | 61.17 | 7.76 | 8.80 |

INFRARED (ν_{cm-1}): 2950; 2908 (νC—H); 2807 (νN—CH₂); 1708 (νC═O); 1656 (νC═C); 1435 (δC—H); 1351 (νC—N); 1258 (νC—O).

### Preparation 48: Ethyl(±)-1-[2-[4-[(tetrahydrofuran-2-yl)methyl]piperazin-1-yl)]ethyl]piperidine-3-carboxylate

The product is obtained according to the procedure of Preparation 20 using 4-[(tetrahydrofuran-2-yl)methyl]piperidine instead of morpholine.

Chromatography over silica gel (CH₂Cl₂/CH₃OH: 95/5) allows 1.2 g of the expected product to be isolated.

Infrared (ν_{cm-1})

2939; 2871; 2810 (νC—H); 1730 (νC═O ester) 1451 (δC—H); 1371 (νC—N); 1300; 1154 (νC—O).

### Preparation 49: Ethyl 1-[2-[4-[2-(dimethylamino)ethyl]piperazin-1-yl)]ethyl]-piperidine-3-carboxylate

The product is obtained according to the procedure of Preparation 20 using N-[2-(dimethylamino)ethyl]piperidine instead of morpholine.

Infrared (ν_{cm-1})

2941; 2810 (νC—H); 1730 (νC═O ester); 1466 (δC—H); 1304; 1154 (νC—O).

### Preparation 50: Methyl(±)-1-[2-(4-(2-methoxyethyl)piperazin-1-yl)ethyl]-piperidine-3-carboxylate

The product is obtained according to the procedure of Preparation 20 using (2-methoxyethyl)piperidine instead of morpholine.

Infrared (ν_{cm-1})

2941; 2809 (νC—H); 1734 (νC═O ester); 1451 (δC—H); 1305 (νC—N); 1155 (νC—O)); 1014.

### Preparation 51: Ethyl 1-[2-[4-(1-methylpiperidin-4-yl)piperazin-1-yl]ethyl]-piperidine-3-carboxylate

The product is obtained according to the procedure of Preparation 20 using (1-methylpiperidin-4-yl)piperazine instead of morpholine.

Infrared (ν_{cm-1})

2936; 2807 (νC—H); 1731 (C═O ester); 1449 (δC—H); 1375 (νC—N); 1152 (νC—O).

### Preparation 52: Ethyl(±)-1-[3-[4-[2-(tert-butyldimethylsilyloxy)ethyl]piperazin-1-yl]propyl]piperidine-3-carboxylate

### Step A: Ethyl(±)-1-[3-[4-(2-hydroxyethyl)piperazin-1-yl]propyl]piperidine-3-carboxylate

The product is obtained according to the procedure of Preparation 20 using (2-hydroxyethyl)piperazine instead of morpholine.

Infrared (ν_{cm-1})

2940; 2810 (νC—H); 1731 (νC═O ester); 1573; 1372; 1154 (νC—O).

### Step B: Ethyl(±)-1-[3-[4-[2-(tert-butyldimethylsilyloxy)ethyl]piperazin-1-yl]propyl]-piperidine-3-carboxylate

1.0 g of the compound of Step A above is dissolved in 10 ml of pyridine, and 0.7 g of TBDMSCl is added. The reaction mixture is stirred at ambient temperature for 12 hours; the solvent is removed, in the presence of toluene, by distilling with the aid of a rotary evaporator. Chromatography over silica gel (CH₂Cl₂/MeOH: 95/5) allows 1.15 g of the expected product to be obtained.

INFRARED (ν_{cm-1})

2935; 2858; 2807 (νC—H); 2477; 1731 (νC═O ester); 1460 (δC—H); 1409; 1372; 1337; 1314; 1278; 1217; 1181.

### Preparation 53: 3-[N-(Indol-1-yl)aminocarbonyl]-1-(4-chlorobutyl)pyridinium bromide

### Step A: 1-(4-Chlorobutyl)-3-(methoxycarbonyl)pyridinium bromide

3.0 g of methyl nicotinate are dissolved in 9 ml of methanol, and then 5 ml of 1-bromo-4-chlorobutane are added. The reaction mixture is heated at reflux for 24 hours. After return to ambient temperature, 25 ml of diethyl ether are added. The oil formed is isolated by drawing off the supernatant and is washed twice with 20 ml of diethyl ether. After drying under reduced pressure, 4.2 g of the expected product are obtained.

### Step B: 3-[N-Indol-1-yl)aminocarbonyl]-1-(4-chlorobutyl)pyridinium bromide

0.28 g of the compound of Preparation 2 is dissolved in 4 ml of anhydrous dichloromethane. The solution is cooled to −20° C. and 2.1 ml of 2M trimethylaluminium solution in hexane are added under argon. After raising the temperature of the reaction mixture to 0° C. over 1 hour 30 minutes, a solution of 0.6 g of the compound of Step A above in 4 ml of anhydrous dichloromethane is added. The reaction mixture is then heated at reflux for 18 hours. After return to ambient temperature, 40 ml of dichloromethane are added and 20% (w/v) aqueous sodium hydroxide solution is added dropwise until no more methane is evolved. The organic phase is separated off, washed successively with 20% (w/v) aqueous sodium hydroxide solution and saturated NaCl solution, dried over Na₂SO₄ and concentrated to a volume of about 10 ml. 50 ml of diethyl ether are added. The solid obtained is separated off by filtration and then rinsed several times with diethyl ether. After drying, 0.53 g of the expected product is obtained.

Melting point: 164° C.

Infrared (ν_{cm-1})

3028; 1625; 1589; 1556; 1443; 1286; 1210; 1174.

### Preparation 54: Ethyl(±)-1-[2-(4-butylpiperazin-1-yl)ethyl]piperidine-3-carboxylate

The product is obtained according to the procedure of Preparation 20 using 1-butylpiperazine instead of morpholine.

Chromatography over silica gel (CH₂Cl₂/CH₃OH: 9/1+0.5% NEt₃) allows 1.2 g of the expected product to be isolated.

Infrared (ν_{cm-1})

2935; 2806 (νC—H); 1732 (νC═O ester); 1450 (δC—H); 1373 (νC—N); 1155 (νC—O).

### Preparation 55: Ethyl(±)-1-(prop-2-ynyl)piperidine-3-carboxylate

The product is obtained according to the procedure of Preparation 11 using propargyl bromide instead of allyl bromide.

Flash chromatography over silica gel (cyclohexane/AcOEt:85/15, then 8/2) allows 500 mg of the expected product to be obtained.

Elemental microanalyses:

| | C % | H % | N % |
|---|---|---|---|
| Calculated: | 67.66 | 8.78 | 7.17 |
| Found: | 67.44 | 8.98 | 7.30 |

INFRARED (ν_{cm-1}): 3291 (ν≡C—H); 2940; 2858; 2806 (νC—H); 1727 (νC═O); 1629 (νC═C); 1468; 1450 (δC—H); 1368; 1310 (νN—C); 1223; 1181 (νC—O).

### Preparation 56: Ethyl(±)-1-[4-(piperidin-1-yl)but-2-en-1-yl]piperidine-3-carboxylate

Under a well-ventilated hood, there are added, successively and at ambient temperature, to a solution of 680 mg of 1,4-dibromobutene in 5 ml of benzene, dropwise, a solution of 500 mg of ethyl nipecotate in 5 ml of benzene and 340 mg of sodium carbonate. The reaction mixture is stirred at ambient temperature for 18 hours, and then 1.26 ml of piperidine are added. The suspension is stirred at ambient temperature for one hour and the reaction mixture is evaporated under reduced pressure. The residue is dissolved in dichloromethane and the organic phase is extracted with aqueous sodium carbonate solution. The aqueous phase is extracted with dichloromethane. The combined organic phases are dried over sodium sulphate, filtered and evaporated under reduced pressure. Flash chromatography over silica gel (CH₂Cl₂CH₃OH: 95/5, then 9/1) allows 260 mg of the expected product to be obtained.

Infrared (ν_{cm-1})

2934; 2854 (νC—H); 2796; 2757 (νN—CH₂); 1731 (νC═O); 1467; 1442 (δC—H); 1368; 1352 (νC—N); 1218; 1180 (νC—O).

### EXAMPLE 1

### N-(1H-indol-1-yl)-1-methyl-1,2,5,6-tetrahydropyridine-3-carboxamide

1.94 g of methyl 1-methyl-1,2,5,6-tetrahydropyridine-3-carboxylate hydrochloride are dissolved in 5 ml of water, and the solution is then made alkaline using potassium carbonate to achieve a pH of 10 and is then saturated with NaCl. The aqueous phase is extracted three times with diethyl ether. The combined organic phases are dried over Na₂SO₄, filtered and then evaporated. 1.3 g of the compound of Preparation 2 are dissolved in 26 ml of anhydrous dichloromethane and then, after cooling to −25° C., 9 ml of a 2M solution of trimethylaluminium in hexane are added. After 1 hour 30 minutes, a solution of 1.27 g of arecoline in 6.5 ml of anhydrous dichloromethane is added at ambient temp-erature. The reaction mixture is refluxed overnight and is then diluted with dichloro-methane and poured into 50 ml of 20% aqueous sodium hydroxide solution. The organic phase is isolated and the aqueous phase is extracted with dichloromethane. The combined organic phases are washed with saturated NaCl solution, dried over sodium sulphate, filtered and then evaporated under reduced pressure. Flash chromatography on silica gel (CH₂Cl₂/MeOH: 95/5 and then 9/1) allows 470 mg of the expected product to be isolated.

Melting point: 194° C.
Elemental microanalyses:

| | C % | H % | N % |
|---|---|---|---|
| Calculated: | 70.56 | 6.71 | 16.46 |
| Found: | 70.35 | 6.80 | 16.36 |

Mass spectrometry (ESI+, m/z): 256.1 (M + H⁺); 278.1 (M + Na⁺).

### EXAMPLE 2

### N-(1H-indol-1-yl)-1-methyl-1,2,3,6-tetrahydropyridine-3-carboxamide

The expected product is obtained in the course of purification of the compound of Example 1. 180 mg of the compound are obtained after taking up the residue in diethyl ether, trituration and filtration over a glass frit.

Melting point: 117° C.
Elemental microanalyses:

| | C % | H % | N % |
|---|---|---|---|
| Calculated: | 70.56 | 6.71 | 16.46 |
| Found: | 70.08 | 6.81 | 16.24 |

### EXAMPLE 3

### N-(1H-indol-1-yl)-1-methyl-1,4,5,6-tetrahydropyridine-3-carboxamide

1 g of the compound of Preparation 2 is dissolved in 10 ml of anhydrous dichloromethane and then the reaction mixture is cooled to −20° C. 5.5 ml of a 2M solution of trimethylaluminium in hexane are added and the reaction mixture is stirred for 1 hour 30 minutes whilst allowing the temperature to increase. A solution of 1.5 g of the compound of Preparation 7 in 5 ml of dichloromethane is added and the reaction mixture is refluxed for 12 hours. The reaction mixture is diluted with dichloromethane and then poured into 20% aqueous sodium hydroxide solution. The organic phase is separated off and washed, first with 20% sodium hydroxide solution and then with saturated NaCl solution. The organic phase is dried over sodium sulphate, filtered and then evaporated under reduced pressure. Flash chromatography on silica gel (dichloromethane) allows 0.640 g of the expected product to be isolated.

Melting point: 207° C.
Elemental microanalyses:

| | C % | H % | N % |
|---|---|---|---|
| Calculated: | 70.56 | 6.71 | 16.46 |
| Found: | 70.40 | 6.70 | 16.35 |

Mass spectrometry (ESI+, m/z): 256 (M + H⁺).

### EXAMPLE 4

### N-(2,3-dihydro-1H-indol-1-yl)-1-methyl-1,4,5,6-tetrahydropyridine-3-carboxamide

### Step A: N-(2,3-dihydro-1H-indol-1-yl)nicotinamide

To 600 mg of the compound of Preparation 2 dissolved in 18 ml of anhydrous dichloromethane there are added, at 0° C., 108 mg of DMAP and 1.9 ml of triethylamine followed by the addition of 955 mg of nicotinoyl chloride hydrochloride. The reaction mixture is stirred overnight at ambient temperature and is then diluted with dichloromethane and then washed with potassium carbonate solution until a pH of 11 is achieved. The aqueous phase is extracted with dichloromethane. The combined organic phases are washed with saturated NaCl solution, dried over Na₂SO₄, filtered and then evaporated under reduced pressure. Flash chromatography on silica gel (CH₂Cl₂ and then CH₂Cl₂/CH₃OH: 96/4 and 95/5) allows 600 mg of the expected product to be isolated.

Infrared (ν_{cm-1})

3224 (νN—H); 3048 (ν═C—H); 2845 (νC—H); 1656 (νC═O); 1590; 1532 (νC═C).

### Step B: 3-[(2,3-Dihydro-1H-indol-1-ylamino)carbonyl]-1-methylpyridinium iodide

420 mg of the compound of Step A above are dissolved in 1.56 ml of iodomethane. A brown oil is formed which is stirred for 24 hours at ambient temperature protected from light. The excess of iodomethane is removed under reduced pressure. 670 mg of the expected product are thereby obtained.

Infrared (ν_{cm-1})

3224 (νN—H); 3048 (ν═C—H); 2845 (νC—H); 1656 (νC═O); 1590; 1532 (νC═C).

### Step C: N-(2,3-dihydro-1H-indol-1-yl)-1-methyl-1,4,5,6-tetrahydropyridine-3-carboxamide

1.1 g of the compound of Step B above are dissolved in 15 ml of methanol, and then 450 μl of triethylamine and 100 mg of PtO₂ are added. The reaction mixture is degassed and then placed under a hydrogen atmosphere (operation repeated twice). The reaction mixture is stirred for one hour at ambient temperature and is then purged with nitrogen. The mixture is filtered over Celite (eluting with methanol) and then the solvents are evaporated off under reduced pressure. Flash chromatography on silica gel (AcOEt) allows 320 mg of the expected product to be isolated.

Melting point: 127° C.
Elemental microanalyses:

| | C % | H % | N % |
|---|---|---|---|
| Calculated: | 70.01 | 7.44 | 16.33 |
| Found: | 69.80 | 7.55 | 16.24 |

### EXAMPLE 5

### 1-Methyl-N-(2-methyl-2,3-dihydro-1H-indol-1-yl)-1,2,5,6-tetrahydro-pyridine-3-carboxamide

The product is obtained according to the procedure of Example 3, using the compound of Preparation 8 instead of the compound of Preparation 2, and arecoline hydrochloride instead of the compound of Preparation 7.

Melting point: 178° C.
Elemental microanalyses:

| | C % | H % | N % |
|---|---|---|---|
| Calculated: | 70.82 | 7.80 | 15.49 |
| Found: | 69.97 | 7.84 | 15.11 |

Mass spectrometry (ESI+, m/z): 272 (M + H⁺)

### EXAMPLE 6

### N-(5-chloro-1H-indol-1-yl)-1-methyl-1,2,5,6-tetrahydropyridine-3-carboxamide

The product is obtained according to the procedure of Example 3, using the compound of Preparation 3 instead of the compound of Preparation 2, and arecoline instead of the compound of Preparation 7.

Melting point: 155° C.
Elemental microanalyses:

| | C % | H % | N % |
|---|---|---|---|
| Calculated: | 62.18 | 5.57 | 14.50 |
| Found: | 62.09 | 5.59 | 14.44 |

### EXAMPLE 7

### N-(5-fluoro-1H-indol-1-yl)-1-methyl-1,2,5,6-tetrahydropyridine-3-carboxamide

The product is obtained according to the procedure of Example 3, using the compound of Preparation 9 instead of the compound of Preparation 2, and arecoline instead of the compound of Preparation 7.

Melting point: 105° C.
Elemental microanalyses:

| | C % | H % | N % |
|---|---|---|---|
| Calculated: | 65.92 | 5.90 | 15.37 |
| Found: | 65.75 | 5.96 | 15.19 |

### EXAMPLE 8

### N-(5-methoxy-1H-indol-1-yl)-1-methyl-1,2,5,6-tetrahydropyridine-3-carboxamide

The product is obtained according to the procedure of Example 3, using the compound of Preparation 10 instead of the compound of Preparation 2, and arecoline instead of the compound of Preparation 7.

Melting point: 162° C.
Elemental microanalyses:

| | C % | H % | N % |
|---|---|---|---|
| Calculated: | 67.35 | 6.71 | 14.72 |
| Found: | 66.76 | 6.85 | 14.73 |

### EXAMPLE 9

### N-(2,3-dimethyl-1H-indol-1-yl)-1-methyl-1,2,5,6-tetrahydropyridine-3-carboxamide

The product is obtained according to the procedure of Example 3, using the compound of Preparation 5 instead of the compound of Preparation 2, and arecoline instead of the compound of Preparation 7.

Melting point: 168° C.
Elemental microanalyses:

| | C % | H % | N % |
|---|---|---|---|
| Calculated: | 72.06 | 7.47 | 14.83 |
| Found: | 71.59 | 7.74 | 14.75 |

### EXAMPLE 10

### 1-Methyl-N-(3-methyl-1H-indol-1-yl)-1,2,5,6-tetrahydropyridine-3-carboxamide

The product is obtained according to the procedure of Example 3, using the compound of Preparation 4 instead of the compound of Preparation 2, and arecoline instead of the compound of Preparation 7.

Melting point: 152° C.
Elemental microanalyses:

| | C % | H % | N % |
|---|---|---|---|
| Calculated: | 71.35 | 7.11 | 15.60 |
| Found: | 71.36 | 7.17 | 15.59 |

### EXAMPLE 11

### N-(1H-indol-1-yl)-1-methyl-1,2,5,6-tetrahydropyridine-4-carboxamide

The product is obtained according to the procedure of Example 3, using methyl 1-methyl-1,2,5,6-tetrahydropyridine-4-carboxylate instead of the compound of Preparation 7.

Melting point: 112° C.
Elemental microanalyses:

| | C % | H % | N % |
|---|---|---|---|
| Calculated: | 70.56 | 6.71 | 16.46 |
| Found: | 70.23 | 6.79 | 16.17 |

### EXAMPLE 12

### 1-Allyl-N-(5-chloro-1H-indol-1-yl)piperidine-3-carboxamide

The product is obtained according to the procedure of Example 3, using the compound of Preparation 3 instead of the compound of Preparation 2, and the compound of Preparation 11 instead of the compound of Preparation 7.

Melting point: 130° C.
Elemental microanalyses:

| | C % | H % | N % |
|---|---|---|---|
| Calculated: | 64.25 | 6.34 | 13.22 |
| Found: | 64.18 | 6.47 | 13.20 |

### EXAMPLE 13

### N-(5-chloro-1H-indol-1-yl)piperidine-3-carboxamide

The product is obtained according to the procedure of Example 3, using the compound of Preparation 3 instead of the compound of Preparation 2, and ethyl nipecotate instead of the compound of Preparation 7.

Melting point: 167° C.
Elemental microanalyses:

| | C % | H % | N % |
|---|---|---|---|
| Calculated: | 60.54 | 5.81 | 15.13 |
| Found: | 60.36 | 5.93 | 15.12 |

Mass spectrometry (EI): 277 (M + H⁺)

### EXAMPLE 14

### 3-[(1H-Indol-1-ylamino)carbonyl]-1-methylpiperidinium chloride

### Step A: N-(1H-indol-1-yl)-1-methylpiperidine-3-carboxamide

The product is obtained according to the procedure of Example 3, using the compound of Preparation 12 instead of the compound of Preparation 7.

Mass spectrometry (ESI+, m/z): 258 (M+H⁺)

### Step B: 3-[(1H-indol-1-ylamino)carbonyl]-1-methylpiperidinium chloride

6 drops of concentrated HCl are added to a solution of 228 mg of the compound of Step A above in 6 ml of anhydrous THF. The solvent is evaporated off and the residue is then washed with diethyl ether, allowing 256 mg of the expected product to be obtained.

Melting point: >250° C.
Elemental microanalyses:

| | C % | H % | N % |
|---|---|---|---|
| Calculated: | 61.32 | 6.86 | 14.30 |
| Found: | 61.14 | 6.99 | 14.16 |

### EXAMPLE 15

### N-(2,3-dihydro-1H-indol-1-yl)-1-methyl-1,2,5,6-tetrahydropyridine-3-carboxamide

The product is obtained according to the procedure of Example 3, using the compound of Preparation 6 instead of the compound of Preparation 2, and arecoline instead of the compound of Preparation 7.

Melting point: 152° C.
Elemental microanalyses:

| | C % | H % | N % |
|---|---|---|---|
| Calculated: | 70.01 | 7.44 | 16.33 |
| Found: | 69.99 | 7.47 | 16.28 |

### EXAMPLE 16

### 1-Methyl-N-(2-methyl-2,3-dihydro-1H-indol-1-yl)piperidine-3-carboxamide

The product is obtained according to the procedure of Example 3, using the compound of Preparation 8 instead of the compound of Preparation 2, and the compound of Preparation 12 instead of the compound of Preparation 7.

Melting point: 133° C.
Elemental microanalyses:

| | C % | H % | N % |
|---|---|---|---|
| Calculated: | 70.30 | 8.48 | 15.37 |
| Found: | 70.19 | 8.56 | 15.23 |

Mass spectrometry (EI): 273 (M + H⁺)

### EXAMPLE 17

### N-(5-chloro-1H-indol-1-yl)-1-propylpiperidine-3-carboxamide

The product is obtained according to the procedure of Example 3, using the compound of Preparation 13 instead of the compound of Preparation 7.

Melting point: 157° C.
Elemental microanalyses:

| | C % | H % | N % |
|---|---|---|---|
| Calculated: | 63.84 | 6.94 | 13.14 |
| Found: | 63.30 | 7.09 | 13.12 |

Mass spectrometry (ESI+, m/z): 320, 322 (M + H⁺)

### EXAMPLE 18

### N-(5-chloro-1H-indol-1-yl)-1-(2-hydroxyethyl)-1,2,5,6-tetrahydro-3-pyridinecarboxamide

### Step A: 1-(2-{[tert-Butyl(dimethyl)silyl]oxy}ethyl)-N-(5-chloro-1H-indol-1-yl)-1,2,5,6-tetrahydro-3-pyridinecarboxamide

1 g of the compound of Preparation 3 is dissolved in 10 ml of anhydrous dichloromethane and then the reaction mixture is cooled to −20° C. 5.5 ml of 2M trimethylaluminium in hexane are added and the reaction mixture is stirred for 1 hour 30 minutes, whilst allowing the temperature to increase. A solution of 1.5 g of the compound of Preparation 15 in 5 ml of dichloromethane is added and the reaction mixture is heated overnight at the reflux of dichloromethane. The reaction mixture is diluted with dichloromethane and is then poured into 20% aqueous sodium hydroxide solution. The organic phase is separated off and is then washed with 20% sodium hydroxide solution and then with saturated NaCl solution. The organic phase is dried over sodium sulphate, filtered and then evaporated under reduced pressure. Flash chromatography on silica gel (AcOEt/cyclohexane:1/1 and then 2/1) allows 1.5 g of the expected product to be isolated.

Infrared (ν_{cm-1})

3244 (νN—H); 2953; 2928; 2856 (νC—H); 1672 (νC═O); 1642; 1520 (νC═C).

### Step B: N-(5-chloro-1H-indol-1-yl)-1-(2-hydroxyethyl)-1,2,5,6-tetrahydro-3-pyridinecarboxamide

6.72 ml of 1M Bu₄NF in THF are added to a solution of 1.46 g of the compound of Step A above in 12 ml of anhydrous THF, and the reaction mixture is then stirred overnight at ambient temperature. The mixture is concentrated under reduced pressure and is then taken up in dichloromethane and washed with water and then with a pH 11 solution of sodium carbonate. The aqueous phases are extracted with dichloromethane. The combined organic phases are dried over sodium sulphate, filtered and then evaporated under reduced pressure. Flash chromatography on silica gel (AcOEt/MeOH:95/5 and then 9/1) allows 920 mg of the expected product to be isolated.

Melting point: 84° C.
Elemental microanalyses:

| | C % | H % | N % |
|---|---|---|---|
| Calculated: | 60.09 | 5.67 | 13.14 |
| Found: | 59.94 | 5.78 | 12.98 |

### EXAMPLE 19

### 1-[2-(Dimethylamino)ethyl]-N-(1H-indol-1-yl)-1,2,5,6-tetrahydro-3-pyridinecarboxamide dihydrochloride

### Step A:1-[2-(Dimethylamino)ethyl]-N-(1H-indol-1-yl)-1,2,56-tetrahydro-3-pyridinecarboxamide

The product is obtained according to the procedure of Step A of Example 18, using the compound of Preparation 2 instead of the compound of Preparation 3, and the compound of Preparation 16 instead of the compound of Preparation 15.

### Step B: 1-[2-(Dimethylamino)ethyl]-N-(1H-indol-1-yl)-1,2,5,6-tetrahydro-3-pyridine-carboxamide dihydrochloride

200 mg of the compound of Step A above are dissolved in 0.5 ml of methanol, and then 285 μl of 4.53M methanolic hydrochloric acid are added. The solution is submerged by ether, and the precipitate formed is filtered off and then rinsed with ether. 210 mg of the expected product are obtained by drying under reduced pressure.

Melting point: 210° C.
Elemental microanalyses:

| | C % | H % | N % |
|---|---|---|---|
| Calculated: | 53.60 | 7.00 | 13.89 |
| Found: | 53.63 | 7.41 | 13.42 |

### EXAMPLE 20

### N-(5-chloro-1H-indol-1-yl)-1-[2-(dimethylamino)ethyl]-1,4,5,6-tetrahydro-3-pyridinecarboxamide

The product is obtained according to the procedure of Step A of Example 18, using the compound of Preparation 18 instead of the compound of Preparation 15.

Melting point: 166° C.
Elemental microanalyses:

| | C % | H % | N % |
|---|---|---|---|
| Calculated: | 62.33 | 6.68 | 16.15 |
| Found: | 62.17 | 6.73 | 16.04 |

Mass spectrometry (ESI+, m/z): 347.1; 349.1 (M + H⁺); 369.1; 371.1 (M + Na⁺).

### EXAMPLE 21

### 1-[2-(Dimethylamino)ethyl]-N-1H-indol-1-yl)-1,4,5,6-tetrahydro-3-pyridinecarboxamide

The product is obtained according to the procedure of Step A of Example 18, using the compound of Preparation 2 instead of the compound of Preparation 3, and the compound of Preparation 18 instead of the compound of Preparation 15.

Melting point: 106° C.
Elemental microanalyses:

| | C % | H % | N % |
|---|---|---|---|
| Calculated: | 69.20 | 7.74 | 17.93 |
| Found: | 68.98 | 7.79 | 17.84 |

Mass spectrometry (ESI+, m/z): 313.1 (M + H⁺); 335.2 (M + Na⁺).

### EXAMPLE 22

### 1-[2-(Dimethylamino)ethyl]-N-(1H-indol-1-yl)-3-piperidinecarboxamide

The product is obtained according to the procedure of Step A of Example 18, using the compound of Preparation 2 instead of the compound of Preparation 3, and the compound of Preparation 19 instead of the compound of Preparation 15.

Melting point: 79° C.
Elemental microanalyses:

| | C % | H % | N % |
|---|---|---|---|
| Calculated: | 68.76 | 8.33 | 17.82 |
| Found: | 68.70 | 8.41 | 17.79 |

Mass spectrometry (ESI+, m/z): 315.2 (M + H⁺); 337.2 (M + Na⁺).

### EXAMPLE 23

### N-(1H-indol-1-yl)-1-[2-(4-morpholinyl)ethyl]-3-piperidinecarboxamide

The product is obtained according to the procedure of Step A of Example 18, using the compound of Preparation 2 instead of the compound of Preparation 3, and the compound of Preparation 20 instead of the compound of Preparation 15.

Melting point: 131° C.
Elemental microanalyses:

| | C % | H % | N % |
|---|---|---|---|
| Calculated: | 67.39 | 7.92 | 15.72 |
| Found: | 67.44 | 8.04 | 15.78 |

### EXAMPLE 24

### N-(1H-indol-1-yl)-1-[2-(1-piperidyl)ethyl]-3-piperidinecarboxamide

The product is obtained according to the procedure of Step A of Example 18, using the compound of Preparation 2 instead of the compound of Preparation 3, and the compound of Preparation 21 instead of the compound of Preparation 15.

Melting point: 113° C.
Elemental microanalyses:

| | C % | H % | N % |
|---|---|---|---|
| Calculated: | 71.15 | 8.53 | 15.80 |
| Found: | 71.01 | 8.54 | 15.75 |

Infrared (ν_{cm-1}): 3108 (νN—H amide + ν═C—H), 2921, 2853 (νC—H), 1690 (νC═O ester).

### EXAMPLE 25

### N-(1H-indol-1-yl)-1-[2-(4-methyl-1-piperazinyl)ethyl]-3-piperidine-carboxamide

The product is obtained according to the procedure of Step A of Example 18, using the compound of Preparation 2 instead of the compound of Preparation 3, and the compound of Preparation 22 instead of the compound of Preparation 15.

Melting point: 117° C.
Elemental microanalyses:

| | C % | H % | N % |
|---|---|---|---|
| Calculated: | 68.26 | 8.46 | 18.95 |
| Found: | 68.14 | 8.49 | 18.88 |

### EXAMPLE 26

### 1-{2-[4-(2-Hydroxyethyl)-1-piperazinyl]ethyl}-N-(1H-indol-1-yl)-3-piperidinecarboxamide

The product is obtained according to the procedure of Step A of Example 18, using the compound of Preparation 2 instead of the compound of Preparation 3, and the compound of Preparation 23 instead of the compound of Preparation 15.

Melting point: 118° C.
Elemental microanalyses:

| | C % | H % | N % |
|---|---|---|---|
| Calculated: | 66.14 | 8.32 | 17.53 |
| Found: | 65.96 | 8.33 | 17.49 |

### EXAMPLE 27

### N-(1H-indol-1-yl)-1-[2-(1-Pyrrolidinyl)ethyl]-3-piperidine-carboxamide

The product is obtained according to the procedure of Step A of Example 18, using the compound of Preparation 2 instead of the compound of Preparation 3, and the compound of Preparation 24 instead of the compound of Preparation 15.

Melting point: 117° C.
Elemental microanalyses:

| | C % | H % | N % |
|---|---|---|---|
| Calculated: | 70.56 | 8.29 | 16.46 |
| Found: | 70.54 | 8.21 | 16.44 |

### EXAMPLE 28

### 1-[2-(1-Azepanyl)ethyl]-N-(1H-indol-1-yl)-3-piperidinecarboxamide

The product is obtained according to the procedure of Step A of Example 18, using the compound of Preparation 2 instead of the compound of Preparation 3, and the compound of Preparation 25 instead of the compound of Preparation 15.

Melting point: >250° C.
Elemental microanalyses:

| | C % | H % | N % |
|---|---|---|---|
| Calculated: | 58.90 | 7.82 | 12.48 |
| Found: | 58.88 | 7.84 | 12.38 |

### EXAMPLE 29

### N-(1H-indol-1-yl)-1-[2-(4-phenyl-1-piperazinyl)ethyl]-3-piperidine-carboxamide trihydrochloride

The product is obtained according to the procedure of Step A of Example 18, using the compound of Preparation 2 instead of the compound of Preparation 3, and the compound of Preparation 26 instead of the compound of Preparation 15.

430 mg of the resulting residue are dissolved in 1.5 ml of anhydrous MeOH, and then 1.65 ml of 2M methanolic HCl solution are added. The trihydrochloride is obtained by precipitation by adding 5 ml of ether, filtration and then drying in vacuo.

Melting point: >250° C.
Elemental microanalyses:

| | C % | H % | N % |
|---|---|---|---|
| Calculated: | 55.87 | 6.85 | 12.52 |
| Found: | 55.84 | 6.83 | 12.45 |

### EXAMPLE 30

### N-(1H-indol-1-yl)-N-({1-[2-(1-piperidyl)ethyl]-3-piperidyl}methyl)-amine trihydrochloride

The product is obtained according to the procedure of Step A of Example 18, using the compound of Preparation 2 instead of the compound of Preparation 3, and the compound of Preparation 27 instead of the compound of Preparation 15.

310 mg of the resulting residue are dissolved in 1.5 ml of anhydrous MeOH, and then 1.5 ml of 2M methanolic HCl solution are added. The trihydrochloride is obtained by precipitation by adding 5 ml of ether, filtration and then drying in vacuo.

Melting point: >250° C.
Elemental microanalyses:

| | C % | H % | N % |
|---|---|---|---|
| Calculated: | 56.06 | 7.84 | 12.45 |
| Found: | 55.88 | 8.02 | 12.29 |

### EXAMPLE 31

### N-(5-chloro-2,3-dihydro-1H-indol-1-yl)-1-(2-hydroxyethyl)-1,4,5,6-tetrahydro-3-pyridinecarboxamide

### Step A: N-(5-chloro-2,3-dihydro-1H-indol-1-yl)nicotinamide

The product is obtained according to the procedure of Step A of Example 4, using the compound of Preparation 28 instead of the compound of Preparation 2.

Infrared (ν_{cm-1})

3265 (νN—H); 3025 (ν═C—H); 2975; 2883 (νC—H); 1652 (νC═O); 1605; 1591; 1578; 1537 (νC═C).

### Step B: 3-{[(5-Chloro-2,3-dihydro-1H-indol-1-yl)amino]carbonyl}-2-(2-hydroxy-ethyl)pyridinium bromide

The product is obtained according to the procedure of Step B of Example 4, using the compound of Step A above and using 2-bromoethanol instead of iodomethane.

Infrared (ν_{cm-1})

3349 (νN—H, vO—H); 3052 (ν═C—H); 2854 (νC—H); 1675 (νC═O); 1634; 1606; 1543 (νC═C).

### Step C: N-(5-chloro-2,3-dihydro-1H-indol-1-yl)-1-(2-hydroxyethyl)-1,4,5,6-tetrahydro-3-pyridinecarboxamide

The product is obtained according to the procedure of Step C of Example 4, using the compound of Step B above.

Melting point: 161° C.
Elemental microanalyses:

| | C % | H % | N % |
|---|---|---|---|
| Calculated: | 59.72 | 6.26 | 13.06 |
| Found: | 59.86 | 6.35 | 13.16 |

### EXAMPLE 32

### N-(2,3-dihydro-1H-indol-1-yl)-1-[2-(dimethylamino)ethyl]-3-piperidinecarboxamide dihydrochloride

### Step A: N-(2,3-dihydro-1H-indol-1-yl)-[2-(dimethylamino)ethyl]-3-piperidine-carboxamide

3 ml of trifluoroacetic acid are added to 500 mg of the compound of Example 22 dissolved in 3 ml of anhydrous THF. The reaction mixture is placed under a current of nitrogen and then a solution of 1 g of sodium cyanoborohydride in 10 ml of THF is added dropwise over 2 hours. The solvents are evaporated off under reduced pressure, the residue is taken up in dichloromethane and water, the mixture is made alkaline with potassium carbonate solution until a pH of 11 is achieved and the aqueous phase is then extracted twice with dichloromethane. The combined organic phases are dried over sodium sulphate, filtered and then evaporated under reduced pressure. The residue is taken up in 13 ml of absolute ethanol, and then 1.5 ml of 2N hydrochloric acid are added. The reaction mixture is refluxed for 5 hours and then evaporated under reduced pressure. The residue is taken up in 20 ml of a 1/1 mixture of dichloromethane and water, the aqueous phase being made alkaline by adding sodium carbonate until a pH of 11 is achieved. The organic phase is separated off, and then the aqueous phase is extracted twice with dichloromethane. The combined organic phases are dried over sodium sulphate, filtered and then evaporated. Flash chromatography on silica gel (AcOEt/MeOH:9/1 and then 8/2 to 6/4) allows 255 mg of the expected product to be isolated.

### Step B: N-(2,3-dihydro-1H-indol-1-yl)-1-[2-(dimethylamino)ethyl]-3-piperidine-carboxamide dihydrochloride

255 mg of the compound of Step A above are dissolved in 4 ml of dichloromethane and then 360 μl of 4.53M methanolic hydrochloric acid are added. The solution is stirred for 5 minutes and then a large excess of ether is added: a precipitate is formed which is filtered off over a glass frit, allowing 270 mg of the expected product to be isolated.

Melting point: 108° C.
Elemental microanalyses:

| | C % | H % | N % |
|---|---|---|---|
| Calculated: | 55.52 | 7.77 | 14.39 |
| Found: | 55.63 | 7.91 | 14.23 |

### EXAMPLE 33

### N-(5-chloro-1H-indol-1-yl)-1-(2-hydroxyethyl)-1,4,5,6-tetrahydro-3-pyridinecarboxamide

The product is obtained according to the procedure of Step A of Example 18, using the compound of Preparation 17 instead of the compound of Preparation 15.

Melting point: 195° C.
Elemental microanalyses:

| | C % | H % | N % |
|---|---|---|---|
| Calculated: | 60.69 | 5.67 | 13.14 |
| Found: | 60.20 | 5.86 | 12.90 |

### EXAMPLE 34

### 1-(2-Hydroxyethyl)-N-(1H-indol-1-yl)-1,4,5,6-tetrahydro-3-pyridinecarboxamide

### Step A: 1-(2-{[tert-Butyl(dimethyl)silyl]oxy}methyl)-N-(1H-indol-1-yl)-1,4,5,6-tetrahydro-3-pyridinecarboxamide

The product is obtained according to the procedure of Step A of Example 18, using the compound of Preparation 2 instead of the compound of Preparation 3, and the compound of Preparation 29 instead of the compound of Preparation 15.

### Step B:1-(2-Hydroxyethyl)-N-(1H-indol-1-yl)-1,4,5,6-tetrahydro-3-pyridine-carboxamide

The product is obtained according to the procedure of Step B of Example 18, using the compound of Step A above.

Melting point: 152° C.
Elemental microanalyses:

| | C % | H % | N % |
|---|---|---|---|
| Calculated: | 67.35 | 6.71 | 14.73 |
| Found: | 67.37 | 6.81 | 14.70 |

Mass spectrometry (ESI+, m/z): 286 (M + H⁺)

### EXAMPLE 35

### N-(Indol-1-yl)-N-[(1-methyl-1,2,5,6-tetrahydropyridin-3-yl)methyl]amine dihydrochloride

### Step A: (Z,E)-(Indol-1-yl)[(pyridin-3-yl)methylene]amine

2.0 g of pyridine-3-carboxaldehyde are dissolved in 100 ml of toluene, and then 3.7 g of the compound of Preparation 2 are added. The mixture is placed in a flask provided with a Dean-Stark apparatus and heated at reflux, with stirring, until the theoretical volume of water has been obtained (18 hours). The reaction mixture is concentrated using a rotary evaporator and the residue is then purified by chromatography over silica gel (CH₂Cl₂/MeOH: 98/2), allowing 3.8 g of the expected compound to be obtained. Melting point: 112° C.

### Step B: 3-[(Z,E)-N-Indol-1-yl)iminomethyl]-1-methylpyridinium iodide

5 ml of iodomethane are added to a solution of 2.0 g of the compound of Step A above in 10 ml of methanol. The reaction mixture is stirred at ambient temperature for 24 hours. 50 ml of diethyl ether are added; the precipitate is separated off by filtration and dried to yield 3.05 g of the expected compound.

Melting point: 218° C.
Elemental microanalyses:

| | C % | H % | N % |
|---|---|---|---|
| Calculated: | 49.60 | 3.89 | 11.57 |
| Found: | 49.62 | 3.98 | 11.48 |

INFRARED (ν_{cm-1}): 3120; 3038; 1633; 1593; 1475; 1450; 1297; 1251; 1158.

### Step C: (Z,E)-(Indol-1-yl)[(1-methyl-1,2,5,6-tetrahydropyridin-3-yl]methylene)]amine

522 mg of NaBH₄ are added in small portions to a solution of 1.0 g of the compound of Step B above in 20 ml of methanol, which solution has been cooled to 0° C. After the addition, the temperature is raised to ambient temperature and then the reaction mixture is stirred for 1 hour. 50 ml of saturated sodium hydrogen carbonate solution are then added and the mixture is extracted with 3×30 ml of dichloromethane. The combined organic phases are extracted successively with sodium hydrogen carbonate solution and saturated NaCl solution. After drying over Na₂SO₄, the solvents are removed in vacuo using a rotary evaporator. Chromatography over silica gel (CH₂Cl₂MeOH: 95/5) allows 0.51 g of the expected product to be obtained.

Elemental microanalyses:

| | C % | H % | N % |
|---|---|---|---|
| Calculated: | 75.28 | 7.16 | 17.56 |
| Found: | 72.02 | 7.35 | 17.75 |

Infrared (ν_{cm-1}): 3104; 3053; 2964; 2960; 2930; 2895; 2846; 2762; 1642; 1614; 1454; 1292; 1255.

### Step D: (Indol-1-yl)[(1-methyl-1,2,5,6-tetrahydropyridin-3-yl)methyl]amine dihydrochloride

0.4 g of LiAlH₄ is added, in small portions and at ambient temperature, to a solution of 0.65 g of the compound of Step C above in 10 ml of diethyl ether. The reaction mixture is stirred at ambient temperature for 2 hours and then 10 ml of water are added. The reaction mixture is extracted with 3×20 ml of dichloromethane; the organic phase is washed with saturated sodium hydrogen carbonate solution, dried over sodium sulphate and filtered, and is then evaporated under reduced pressure. The residue, which is purified by means of chromatography over silica gel (CH₂Cl₂/MeOH: 95/5±0.5% NEt₃), allows 0.56 g of the base to be obtained. 100 mg of the base are dissolved in 0.5 ml of anhydrous MeOH, and then 0.45 ml of 2M methanolic HCl solution is added. The dihydrochloride precipitates out on addition of 5 ml of diethyl ether and is separated off by filtration and then dried in vacuo.

Melting point: 152° C.
Elemental microanalyses:

| | C % | H % | N % |
|---|---|---|---|
| Calculated: | 54.22 | 6.98 | 12.65 |
| Found: | 54.15 | 7.02 | 12.48 |

Infrared (ν_{cm-1}): 2956; 2437; 2024; 1437; 1376; 1258.

### EXAMPLE 36

### 1-Benzyl-N-(5-chloro-1H-indol-1-yl)-3-piperidinecarboxamide

The product is obtained according to the procedure of Step A of Example 18, using the compound of Preparation 31 instead of the compound of Preparation 15.

Melting point: 197° C.
Elemental microanalyses:

| | C % | H % | N % |
|---|---|---|---|
| Calculated: | 68.56 | 6.03 | 11.42 |
| Found: | 68.45 | 6.25 | 11.31 |

### EXAMPLE 37

### 3-{[(5-Chloro-1H-indol-1-yl)amino]carbonyl}-1-methylpiperidinium hydrochloride

### Step A: N-(5-chloro-1H-indol-1-yl)-1-methyl-3-piperidinecarboxamide

The product is obtained according to the procedure of Step A of Example 18, using the compound of Preparation 12 instead of the compound of Preparation 15.

Infrared (ν_{cm-1})

3117, 3081, 2937, 2464, 1697

### Step B: 3-{[(5-Chloro-1H-indol-1-yl)amino]carbonyl}-1-methylpiperidinium hydrochloride

6 drops of concentrated HCl are added to a solution of 228 mg of the compound of Step A above in 6 ml of anhydrous THF. The solvent is evaporated off and the residue is then washed with ethyl ether, allowing 256 mg of the expected product to be obtained.

Melting point: >250° C.
Elemental microanalyses:

| | C % | H % | N % |
|---|---|---|---|
| Calculated: | 54.89 | 5.83 | 12.80 |
| Found: | 54.87 | 5.98 | 12.83 |

Mass spectrometry (ESI+, m/z): 292 (M + H⁺)

### EXAMPLE 38

### (3R,4S)-3-(4-Fluorophenyl)-N-(1H-indol-1-yl)-1-methylpiperidine-4-carboxamide

590 mg of the compound of Preparation 2 are dissolved in 6 ml of anhydrous dichloromethane and then, after cooling to −20° C., 4.1 ml of 2M trimethylaluminium in hexane are added. The reaction mixture is stirred for 1 hour 30 minutes without controlling the temperature. 930 mg of the compound of Preparation 32 in 4 ml of anhydrous dichloromethane are added and the solution is then stirred for 18 hours at the reflux of dichloromethane. The solution is diluted with dichloromethane and then poured into 20% aqueous sodium hydroxide solution. The organic phase is separated off and is then washed with 20% aqueous hydroxide solution and with saturated NaCl solution. The organic phase is dried over sodium sulphate, filtered and then evaporated under reduced pressure. The residue is purified by flash chromatography on silica gel (CH₂Cl₂/MeOH: 97/3 and then 95/5 and 9/1). The expected product is obtained by precipitation in the presence of ether.

Melting point: 134° C.

Mass spectrometry (ESI+, m/z): 352.1 (M+H⁺)

### EXAMPLE 39

### (3R,4R)-3-(4-Fluorophenyl)-N-(1H-indol-1-yl)-1-methylpiperidine-4-carboxamide

The expected product is obtained during purification of the compound of Example 38.

Melting point: 205° C.
Elemental microanalyses:

| | C % | H % | N % |
|---|---|---|---|
| Calculated: | 71.77 | 6.31 | 11.96 |
| Found: | 71.05 | 6.37 | 11.86 |

Mass spectrometry (ESI +, m/z): 352.1 (M + H⁺); 374.2 (M + Na⁺)

### EXAMPLE 40

### tert-Butyl 4-(2-{3-[(1H-indol-1-ylamino)carbonyl]-1-piperidyl}-ethyl)piperazine-1-carboxylate

The product is obtained according to the procedure of Step A of Example 18, using the compound of Preparation 2 instead of the compound of Preparation 3, and the compound of Preparation 33 instead of the compound of Preparation 15.

Melting point: 48° C.
Elemental microanalyses:

| | C % | H % | N % |
|---|---|---|---|
| Calculated: | 65.91 | 8.19 | 15.37 |
| Found: | 65.34 | 8.21 | 15.00 |

### EXAMPLE 41

### 1-[3(Dimethylammonium)propyl]-3-[(1H-indol-1-ylamino)-carbonyl]piperidinium dihydrochloride

The product is obtained according to the procedure of Example 37, using the compound of Preparation 2 instead of the compound of Preparation 3, and the compound of Preparation 34 instead of the compound of Preparation 12.

Melting point: >250° C.
Elemental microanalyses:

| | C % | H % | N % |
|---|---|---|---|
| Calculated: | 54.41 | 7.69 | 13.36 |
| Found: | 54.45 | 7.71 | 13.41 |

### EXAMPLE 42

### N-(1H-indol-1-yl)-1-[3-(1-piperidyl)propyl]-3-piperidine-carboxamide

The product is obtained according to the procedure of Step A of Example 18, using the compound of Preparation 2 instead of the compound of Preparation 3, and the compound of Preparation 38 instead of the compound of Preparation 15.

Melting point: 95° C.
Elemental microanalyses:

| | C % | H % | N % |
|---|---|---|---|
| Calculated: | 68.36 | 8.86 | 14.44 |
| Found: | 68.30 | 8.94 | 14.24 |

### EXAMPLE 43

### N-(1H-indol-1-yl)-1-[3-(4-methyl-1-piperazinyl)propyl]-3-piperidine-carboxamide

The product is obtained according to the procedure of Step A of Example 18, using the compound of Preparation 2 instead of the compound of Preparation 3, and the compound of Preparation 39 instead of the compound of Preparation 15.

Elemental microanalyses:

| | C % | H % | N % |
|---|---|---|---|
| Calculated: | 68.90 | 8.67 | 18.00 |
| Found: | 68.60 | 8.71 | 18.15 |

### EXAMPLE 44

### N-(5-chloro-1H-indol-1-yl)-1-(2-hydroxyethyl)-3-piperidine carboxamide

### Step A: 1-(2-{[tert-Butyl(dimethyl)silyl]oxy}ethyl)-N-(5-chloro-1H-indol-1-yl)-3-piperidinecarboxamide

The product is obtained according to the procedure of Step A of Example 18, using the compound of Preparation 40 instead of the compound of Preparation 15.

Infrared (ν_{cm-1})

3225 (νN—H); 2930; 2856 (νC—H); 1677 (νC═O); 1517 (νC═C).

### Step B.: N-(5-chloro-1H-indol-1-yl)-1-(2-hydroxyethyl)-3-piperidinecarboxamide

The product is obtained according to the procedure of Step B of Example 18, using the compound of Step A above.

Melting point: 125° C.
Elemental microanalyses:

| | C % | H % | N % |
|---|---|---|---|
| Calculated: | 59.72 | 6.55 | 13.06 |
| Found: | 59.02 | 6.26 | 12.79 |

Mass spectrometry (ESI +, m/z): 322.1; 324.1 (M + H⁺); 344.1; 346.1 (M + Na⁺).

### EXAMPLE 45

### 1-(3-Hydroxypropyl)-N-(1H-indol-1-yl)-3-piperidinecarboxamide

### Step A: 1-(3-{[tert-Butyl(dimethyl)silyl]oxy}propyl)-N-(1H-indol-1-yl)-3-piperidine-carboxamide

The product is obtained according to the procedure of Step A of Example 18, using the compound of Preparation 2 instead of the compound of Preparation 3, and the compound of Preparation 41 instead of the compound of Preparation 15.

Melting point: 94° C.

Infrared (ν_{cm-1})

2951; 2927; 2855 (νC—H); 1706 (νC═O); 1614; 1584 (νC═C).

### Step B: 1-(3-Hydroxypropyl)-N-(1H-indol-1-yl)-3-piperidinecarboxamide

The product is obtained according to the procedure of Step B of Example 18, using the compound of Step A above.

Melting point: 51° C.
Elemental microanalyses:

| | C % | H % | N % |
|---|---|---|---|
| Calculated: | 67.75 | 7.69 | 13.94 |
| Found: | 67.18 | 7.82 | 13.49 |

### EXAMPLE 46

### N-(Indol-1-yl)-1-[2-(4-methylpiperazin-1-yl)ethyl]-1,2,5,6-tetrahydropyridine-3-carboxamide

The product is obtained according to the procedure of Example 3 using the compound of Preparation 43 instead of the compound of Preparation 7.

Flash chromatography over silica gel (CH₂Cl₂/CH₃OH: 95/5, then 9/1 and 8/2) allows 380 mg of the expected product to be isolated.

Melting point: 130° C.
Elemental microanalyses:

| | C % | H % | N % |
|---|---|---|---|
| Calculated: | 68.63 | 7.95 | 19.06 |
| Found: | 68.49 | 8.09 | 18.93 |

Infrared (ν_{cm-1}): 3054 (ν═C—H); 2935 (νC—H); 2795 (νN—CH₃); 1681 (νC═O); 1646; 1614 (νC═C); 1521 (δN—H); 1458 (δC—H); 1372 (νC—N).

### EXAMPLE 47

### N-(5-Chloroindol-1-yl)-1-[2-(4-methylpiperazin-1-yl)ethyl]-1,2,5,6-tetrahydropyridine-3-carboxamide

To a solution of 782 mg of the compound of Preparation 3 in 12 ml of anhydrous dichloromethane, previously cooled to −20° C., there are added 4.30 ml of a 2M solution of trimethylaluminium in hexane. The solution is stirred under nitrogen for 1 hour 30 minutes allowing the temperature to gradually come back up to ambient temperature. 1.05 g of a solution of the compound of Preparation 43 in 8 ml of anhydrous dichloromethane are then added. The mixture is heated at reflux under nitrogen for 16 hours. To the reaction mixture, cooled to 0° C., there are added 100 ml of 1M aqueous HCl solution (slow addition at the start), and then 250 ml of water. A first extraction using 3×100 ml of dichloromethane removes the excess of N-amino-5-chloroindole. The aqueous phase is made alkaline using saturated sodium carbonate solution until a pH of 10 is achieved, and it is extracted using 3×100 ml of dichloromethane. The organic phase is dried over sodium sulphate, filtered and concentrated under reduced pressure. Chromatography on a silica column (NH₄OH/MeOH/CH₂Cl₂:1/1/98 to 2/18/80), followed by recrystallisation from 15 ml of a mixture of iPrOH/AcOEt (1/2), allows 470 mg of the expected product to be obtained.

Melting point: 157° C.
Elemental microanalyses:

| | C % | H % | N % |
|---|---|---|---|
| Calculated: | 62.75 | 7.02 | 17.42 |
| Found: | 62.50 | 6.92 | 17.23 |

Mass spectrometry (ESI +, m/z): 402 (M + 1).
Infrared (ν_{cm-1}): 2946 to 2814 (νCH); 1681 (νCO); 1650; 1531; 1465.

### EXAMPLE 48

### N-(5-Methoxyindol-1-yl)-1-[2-(4-methylpiperazin-1-yl)ethyl]-1,2,5,6-tetrahydropyridine-3-carboxamide

The product is obtained according to the procedure of Example 47 using the compound of Preparation 10 instead of the compound of Preparation 3.

Chromatography on a silica column (NH₄OH/MeOH/CH₂Cl₂: 1/1/98 to 2/18/80) followed by trituration in 15 ml of diethyl ether for 15 minutes and followed by crystallisation from 10 ml of a mixture of AcOEt/Et₂O (1/2) allows 820 mg of the expected product to be obtained.

Melting point: 121° C.
Elemental microanalyses:

| | C % | H % | N % |
|---|---|---|---|
| Calculated: | 66.47 | 7.86 | 17.62 |
| Found: | 65.98 | 8.07 | 17.33 |

Mass spectrometry (ESI +, m/z): 398 (M + 1).
Infrared (ν_{cm-1}): 3216 (νNH); 2938 to 2788 (νCH); 1669 (νCO); 1636; 1514; 1477.

### EXAMPLE 49

### N-(5-Methoxyindol-1-yl)-1-[2-(4-methylpiperazin-1-yl)ethyl]-1,2,5,6-tetrahydropyridine-5-carboxamide

The product is obtained in the course of purification of the compound of Example 48. Recrystallisation from 5 ml of diisopropyl ether allows 138 mg of the expected product to be obtained.

Melting point: 127° C.
Elemental microanalyses:

| | C % | H % | N % |
|---|---|---|---|
| Calculated: | 66.47 | 7.86 | 17.62 |
| Found: | 66.41 | 8.02 | 17.41 |

Mass spectrometry (ESI +, m/z): 398 (M + 1).
Infrared (ν_{cm-1}): 3102 (νNH); 2932 and 2799 (νCH); 1684 (νCO); 1480; 1445.

### EXAMPLE 50

### N-(5-Methylindol-1-yl)-1-[2-(4-methylpiperazin-1-yl)ethyl]-1,2,5,6 tetrahydropyridine-3-carboxamide

The product is obtained according to the procedure of Example 47 using the compound of Preparation 44 instead of the compound of Preparation 3.

Chromatography on a silica column (NH₄OH/MeOH/CH₂Cl₂: 1/1/98 to 2/18/80) followed by trituration in 15 ml of diethyl ether for 15 minutes allows 1.16 g of the expected product to be obtained.

Melting point: 140° C.
Elemental microanalyses:

| | C % | H % | N % |
|---|---|---|---|
| Calculated: | 69.26 | 8.19 | 18.36 |
| Found: | 68.85 | 8.41 | 17.96 |

Mass spectrometry (ESI +, m/z): 382 (M + 1).
Infrared (ν_{cm-1}): 2945 to 2816 (νCH); 1681 (νCO); 1651; 1533; 1466.

### EXAMPLE 51

### N-(5-Methylindol-1-yl)-1-[2-(4-methylpiperazin-1-yl)ethyl]-1,2,5,6-tetrahydropyridine-5-carboxamide

The product is obtained in the course of purification of the compound of Example 50. Recrystallisation from 5 ml of diisopropyl ether allows 120 mg of the expected product to be obtained.

Melting point: 131° C.
Elemental microanalyses:

| | C % | H % | N % |
|---|---|---|---|
| Calculated: | 69.26 | 8.19 | 18.36 |
| Found: | 69.12 | 8.41 | 18.18 |

Mass spectrometry (ESI +, m/z): 382 (M + 1).
Infrared (ν_{cm-1}): 3141 (νNH); 2934 to 2766 (νCH); 1694 (νCO); 1515; 1457.

### EXAMPLE 52

### N-(Indol-1-yl)-1-[2-(4-(2-hydroxyethyl)piperazin-1-yl)ethyl]-1,2,5,6-tetrahydropyridine-3-carboxamide

### Step A: N-(Indol-1-yl)-1-[2-[4-[2-(tert-butyldimethylsilanoxy)ethyl]piperazin-1-yl]-ethyl]-1,2,5,6-tetrahydropyridine-3-carboxamide

To a solution of 200 mg of the compound of Preparation 2 in 5 ml of anhydrous dichloromethane, after cooling to −20° C., there are added 1.35 ml of a 2M solution of trimethylaluminium in hexane. The solution is stirred for 1 hour 30 minutes without controlling the temperature and then 500 mg of the compound of Preparation 45 in 3 ml of anhydrous dichloromethane are added at ambient temperature. The mixture is heated at reflux for 16 hours and, after cooling, diluted with dichloromethane and it is poured into 20% aqueous sodium hydroxide solution. The organic phase is collected and the aqueous phase is extracted with dichloromethane. The combined organic phases are dried over sodium sulphate, filtered and evaporated under reduced pressure. Flash chromatography over silica gel (CH₂Cl₂/CH₃OH: 9/1, then 8/2) allows 400 mg of the expected product to be obtained.

Infrared (ν_{cm-1})

3244 (νN—H) 2949; 2929; 2855 (νC—H); 2811 (νN—CH₂); 1674 (νC═O); 1644 (νC═C) 1521; 1505 (δN—H); 1460 (δC—H); 1360 (νC—N); 1256; 1223 (νC—O).

### Step B: N-(Indol-1-yl)-1-[2-[4-(2-hydroxyethyl)piperazin-1-yl]ethyl]-1,2,5,6-tetrahydropyridine-3-carboxamide

To a solution of 300 mg of the compound of Step A above in 4 ml of anhydrous THF there are added 1.17 ml of a 1 M solution of tetrabutylammonium fluoride in THF. The mixture is stirred for 12 hours at ambient temperature, and then the solution is concentrated under reduced pressure. After taking up the residue in dichloromethane, the organic phase is extracted with aqueous sodium hydroxide solution; the aqueous phase is extracted with dichloromethane. The combined organic phases are dried over sodium sulphate, filtered and evaporated under reduced pressure. Flash chromatography over silica gel (AcOEt/MeOH:7/3±0.1% triethylamine) allows 180 mg of the expected product to be obtained.

Melting point: 113° C.
Elemental microanalyses:

| | C % | H % | N % |
|---|---|---|---|
| Calculated: | 63.59 | 8.00 | 16.85 |
| Found: | 64.12 | 8.08 | 16.49 |

Infrared (ν_{cm-1}): 3192 (νN—H); 2939 (νC—H); 2815 (νN—CH₂); 1674 (νC═O); 1644 (νC═C); 1523 (δN—H); 1459 (δC—H); 1353 (νC—N); 1270; 1223 (νC—O).

### EXAMPLE 53

### N-(Indol-1-yl)-1-(2-piperidin-1-yl-ethyl)-1,2,5,6-tetrahydropyridine-3-carboxamide

The product is obtained according to the procedure of Example 3 using the compound of Preparation 46 instead of the compound of Preparation 7.

Flash chromatography over silica gel (CH₂Cl₂/CH₃OH: 9/1, then 8/2) allows 240 mg of the expected product to be isolated.

Melting point: 65° C.
Elemental microanalyses:

| | C % | H % | N % |
|---|---|---|---|
| Calculated: | 69.78 | 8.09 | 15.50 |
| Found: | 69.98 | 8.17 | 15.40 |

Infrared (ν_{cm-1}): 3238 (νN—H); 2931 (νC—H); 2788 (νN—CH₂); 1672 (νC═O); 1643 (νC═C); 1521 (δN—H); 1459 (δC—H); 1370 (νC—N).

### EXAMPLE 54

### N-(Indol-1-yl)-1-[2-[3-(ethoxycarbonyl)-1,2,5,6-tetrahydropyridin-1-yl]ethyl]-1,2,5,6-tetrahydropyridine-3-carboxamide

The product is obtained according to the procedure of Example 3 using the compound of Preparation 47 instead of the compound of Preparation 7.

Flash chromatography over silica gel (AcOEt, then AcOEt/CH₃OH: 95/5) allows 180 mg of the expected product to be isolated.

Melting point: 82° C.
Elemental microanalyses:

| | C % | H % | N % |
|---|---|---|---|
| Calculated: | 67.63 | 6.91 | 13.72 |
| Found: | 67.41 | 7.09 | 13.63 |

Infrared (ν_{cm-1}): 3265 (νN—H); 2948; 2915; 2802 (νC—H); 1708; 1673 (νC═O); 1646; 1614; 1519 (νC═C); 1459; 1436 (δC—H); 1371; 1351 (νC—N); 1261; 1223; 1194 (νC—O).

### EXAMPLE 55

### (±)N—(Indol-1-yl)-1-[2-[4-[(tetrahydrofuran-2-yl)methyl]piperazin-1-yl)]ethyl]piperidine-3-carboxamidyl trihydrochloride

0.49 g of the compound of Preparation 48 is dissolved in 9 ml of anhydrous dichloromethane. The solution is cooled to −20° C., and then 3.5 ml of a 2M solution of trimethylaluminium in hexane are added under argon. After bringing the temperature of the reaction mixture back up to 0° C. over 1 hour 30 minutes, a solution of 1.1 g of the compound of Preparation 48 in 6 ml of anhydrous dichloromethane is added. The reaction mixture is then heated at reflux for 18 hours. After cooling, 50 ml of dichloromethane are added; then 20% (w/v) aqueous sodium hydroxide solution is added dropwise until no more methane is evolved. The organic phase is separated off, washed successively with 20% (w/v) aqueous sodium hydroxide solution and with saturated NaCl solution, then dried over Na₂SO₄ and concentrated. Chromatography over silica gel (CH₂Cl₂/MeOH: 9/1+0.5% NEt₃) allows 0.46 g of the expected amide to be obtained. 150 mg of the amide are dissolved in 1.0 ml of anhydrous MeOH; 0.6 ml of 2M methanolic HCl solution is added to the solution. The expected product is precipitated out by adding 5 ml of diethyl ether; it is then separated off by filtration and dried in vacuo.

Melting point: >250° C.
Elemental microanalyses:

| | C % | H % | N % |
|---|---|---|---|
| Calculated: | 54.70 | 7.34 | 12.76 |
| Found: | 55.02 | 7.48 | 12.70 |

Infrared (ν_{cm-1}): 3385; 2975; 2411; 1694; 1531; 1458; 1325.

### EXAMPLE 56

### Ethyl(±)—N-(indol-1-yl)-1-[2-[4-[2-(dimethylamino)ethyl]piperazin-1-yl)]ethyl]piperidine-3-carboxylate tetrahydrochloride

The product is obtained according to the procedure of Example 55 using the compound of Preparation 49 instead of the compound of Preparation 48.

Chromatography over silica gel (CH₂Cl₂/MeOH: 9/1 to 7/3±0.5% NEt₃) allows 0.59 g of the expected amide to be obtained. 130 mg of the amide are dissolved in 0.5 ml of anhydrous MeOH; 0.7 ml of 2M methanolic HCl solution is added to the solution. The expected product is obtained by precipitation on addition of 5 ml of diethyl ether; it is separated off by filtration and dried in vacuo.

Melting point: >250° C.
Elemental microanalyses:

| | C % | H % | N % |
|---|---|---|---|
| Calculated: | 50.36 | 7.40 | 14.68 |
| Found: | 50.15 | 7.48 | 14.40 |

Infrared (ν_{cm-1}): 2938; 2815; 1689; 1460; 1396; 1315.

### EXAMPLE 57

### (±)—N-(Indol-1-yl)-1-[2-[4-(2-methoxyethyl)piperazin-1-yl)]ethyl]-piperidine-3-carboxamide trihydrochloride

The product is obtained according to the procedure of Example 55 using the compound of Preparation 50 instead of the compound of Preparation 48.

Chromatography over silica gel (CH₂Cl₂/MeOH: 90/10) allows 0.22 g of the expected amide to be obtained. 200 mg of the amide are dissolved in 1.0 ml of anhydrous MeOH; 0.8 ml of 2M methanolic HCl solution is added to the solution. The expected product is obtained by precipitation on addition of 5 ml of diethyl ether; it is separated off by filtration and dried in vacuo.

Melting point: >250° C.
Elemental microanalyses:

| | C % | H % | N % |
|---|---|---|---|
| Calculated: | 52.83 | 7.32 | 13.39 |
| Found: | 52.74 | 7.39 | 13.32 |

Infrared (ν_{cm-1}): 3381; 2981; 2503; 2299; 1689; 1656; 1449; 1325.

### EXAMPLE 58

### (±)—N-(Indol-1-yl)-1-[2-[4-(1-methylpiperidin-4-yl)piperazin-1-yl)]ethyl]piperidine-3-carboxamide tetrahydrochloride

The product is obtained according to the procedure of Example 55 using the compound of Preparation 51 instead of the compound of Preparation 48.

Chromatography over silica gel (CH₂Cl₂/MeOH: 8/2±0.5% NEt₃) allows 0.51 g of the expected amide to be obtained. 227 mg of the amide are dissolved in 1.0 ml of anhydrous MeOH; 1.1 ml of 2M methanolic HCl solution are added to the solution. The expected product is obtained by precipitation on addition of 5 ml of diethyl ether; it is separated off by filtration and dried in vacuo.

Melting point: >250° C.
Elemental microanalyses:

| | C % | H % | N % |
|---|---|---|---|
| Calculated: | 52.18 | 7.41 | 14.04 |
| Found: | 52.03 | 7.41 | 13.94 |

Infrared (ν_{cm-1}): 3502; 3126; 2980; 2410; 1696 (νC═O amide); 1543; 1473; 1458.

### EXAMPLE 59

### (±)—N-(Indol-1-yl)-1-[3-[4-(2-hydroxyethyl)piperazin-1-yl)]propyl]-piperidine-3-carboxamide trihydrochloride

### Step A: (±)—N-(Indol-1-yl)-1-[3-[4-(2-tert-butyldimethylsilyloxyethyl)piperazin-1-yl)]-propyl]piperidine-3-carboxamide

The product is obtained according to the procedure of Example 55 using the compound of Preparation 52 instead of the compound of Preparation 48.

Chromatography over silica gel (CH₂Cl₂MeOH: 9/1 to 8/2±0.5% NEt₃) allows 0.89 g of the expected product to be obtained.

Infrared (ν_{cm-1})

2934; 2856; 2810; 1682; 1460; 1361; 1253; 1155; 1099.

### Step B: (±)—N-(Indol-1-yl)-1-[3-[4-(2-hydroxyethyl)piperazin-1-yl)]propyl]piperidine 3-carboxamide trihydrochloride

0.35 g of the product of Step A above is dissolved in 5 ml of ethanol 95%. 500 μl of 12M HCl solution are added at ambient temperature. The solution is then heated at 60° C. for 2 hours. After cooling to ambient temperature, the suspension is filtered over a glass frit; the solid obtained is washed several times with ethanol and dried, allowing 0.29 g of the expected product to be obtained.

Melting point: >250° C.
Elemental microanalyses:

| | C % | H % | N % |
|---|---|---|---|
| Calculated: | 53.83 | 7.32 | 13.39 |
| Found: | 53.37 | 7.41 | 13.20 |

Infrared (ν_{cm-1}): 3266; 3155; 2978; 2688; 2503; 2436; 1713; 1515; 1499; 1460.

### EXAMPLE 60

### (±)—N-(Indol-1-yl)-1-[4-(4-methylpiperazin-1-yl)butyl]piperidine-3-carboxamide trihydrochloride

### Step A: 3-[N-(Indol-1-yl)aminocarbonyl]-1-[4-(4-methylpiperazin-1-yl)butyl]-pyridinium bromide

0.6 g of the compound of Preparation 53 and 500 μl of N-methylpiperazine are dissolved in 8 ml of ethanol 70%. The reaction mixture is stirred at 50° C. for 18 hours. An additional 1 ml of N-methylpiperazine is added twice in the course of the next 24 hours under the same conditions. The solvents and the excess of N-methylpiperazine are removed by distillation using a rotary evaporator. The crude residue is used in the next Step without further purification.

### Step B: N-(Indol-1-yl)-1-[4-(4-methylpiperazin-1-yl)butyl]-1,4,5,6-tetrahydro-pyridine-3-carboxamide

The compound of Step A above is dissolved in 9 ml of a mixture of ethyl acetate/ethanol (6:3); 100 mg of palladium-on-carbon (10%) are added. The suspension is degassed with stirring in vacuo and is then placed under a hydrogen atmosphere. The reaction mixture is held for 18 hours under the hydrogen atmosphere, is then purged and is placed under a nitrogen atmosphere. The reaction mixture is filtered over Celite and the filtrate is concentrated. Chromatography over silica gel (CH₂Cl₂MeOH: 9/1 to 8/2±0.5% NEt₃) allows 0.29 g of the expected product to be obtained.

Infrared (ν_{cm-1})

2933; 1615; 1574; 1497; 1355; 1293; 1181.

### Step C: (±)(Indol-1-yl)-1-[4-(4-methylpiperazin-1-yl)butyl]piperidine-3-carboxamide trihydrochloride

To a solution of 295 mg of the compound of Step B above in 8 ml of a mixture of acetic acid/methanol (6:2) there are added, in small portions and at ambient temperature, 200 mg of sodium cyanoborohydride. The reaction mixture is stirred for 3 hours at ambient temperature. 20 ml of 10% sodium hydroxide solution and 20 ml of dichloromethane are then added. The organic phase is washed 3 times with 10% sodium hydroxide solution, dried over sodium sulphate, filtered and evaporated under reduced pressure. Chromatography over silica gel (CH₂Cl₂/MeOH: 8/2±0.5% NEt₃) allows 0.14 g of the amide to be obtained. 100 mg of the amide are dissolved in 0.5 ml of anhydrous MeOH; 0.75 ml of 2M methanolic HCl solution is added to the solution. The expected product is obtained by precipitation on addition of 5 ml of diethyl ether; it is separated off by filtration and dried in vacuo.

Melting point: >250° C.
Elemental microanalyses:

| | C % | H % | N % |
|---|---|---|---|
| Calculated: | 54.49 | 7.56 | 13.82 |
| Found: | 54.60 | 7.80 | 13.61 |

Infrared (ν_{cm-1}): 3382; 2948; 2401; 1687; 1528; 1458; 1315.

### EXAMPLE 61

### (±)—N-(Indol-1-yl)-1-[4-[4-(2-hydroxyethyl)piperazin-1-yl)]butyl]-piperidine-3-carboxamide trihydrochloride

Step A: 3-[N-(Indol-1-yl)aminocarbonyl]-1-[4-([4-(2-hydroxyethyl)piperazin-1-yl)butyl]pyridinium bromide

The product is obtained according to the procedure of Step A of Example 60 using the compound 1-(2-hydroxyethyl)piperazine instead of N-methylpiperazine.

### Step B: N-(Indol-1-yl)-1-[4-[4-(2-hydroxyethyl)piperazin-1-yl)]butyl]-1,4,5,6-tetrahydropyridine-3-carboxamide

The product is obtained according to the procedure of Step B of Example 60 using the compound of Step A above.

Chromatography over silica gel (CH₂Cl₂MeOH: 8/2 to 7/3±0.5% NEt₃) allows 0.12 g of the expected product to be obtained.

### Step C: (±)—N-(Indol-1-yl)-1-[4-[4-(2-hydroxyethyl)piperazin-1-yl)]butyl]piperidine-3-carboxamide trihydrochloride

The product is obtained according to the procedure of Step C of Example 60 using the compound of Step B above.

Melting point: >250° C.
Elemental microanalyses:

| | C % | H % | N % |
|---|---|---|---|
| Calculated: | 53.68 | 7.51 | 13.04 |
| Found: | 53.42 | 7.61 | 12.89 |

### EXAMPLE 62

### (±)—N-(Indol-1-yl)-1-[2-(4-butylpiperazin-1-yl)]ethyl]piperidine-3-carboxamide trihydrochloride

The product is obtained according to the procedure of Example 55 using the compound of Preparation 54 instead of the compound of Preparation 48.

Chromatography over silica gel (CH₂Cl₂/MeOH: 9/1±0.5% NEt₃) allows 0.82 g of the expected amide to be obtained. 600 mg of the amide are dissolved in 2.0 ml of anhydrous MeOH; 2.3 ml of 2M methanolic HCl solution are added to the solution. The expected product is obtained by precipitation on addition of 10 ml of diethyl ether; it is separated off by filtration and dried in vacuo.

Melting point: >250° C.
Elemental microanalyses:

| | C % | H % | N % |
|---|---|---|---|
| Calculated: | 55.33 | 7.74 | 13.44 |
| Found: | 55.12 | 7.91 | 13.26 |

Infrared (ν_{cm-1}): 3371; 2959; 1686; 1535; 1459; 1372; 1314.

### EXAMPLE 63

### (±)—N-(Indol-1-yl)-1-allylpiperidine-3-carboxamide

The product is obtained according to the procedure of Example 3 using the compound of Preparation 11 instead of the compound of Preparation 7.

Flash chromatography over silica gel (AcOEt) allows 930 mg of the expected product to be obtained.

Melting point: 92° C.
Elemental microanalyses:

| | C % | H % | N % |
|---|---|---|---|
| Calculated: | 72.06 | 7.47 | 14.83 |
| Found: | 71.70 | 7.73 | 14.61 |

Infrared (ν_{cm-1}): 3198 (νN—H); 3032 (ν═C—H); 2940 (νC—H); 2792 (νN—CH₂); 1687 (νC═C); 1667 (νC═O); 1615 (νC═C); 1542 (δN—H); 1462; 1421 (δC—H); 1378; 1362 (νC—N).

### EXAMPLE 64

### (±)—N-(Indol-1-yl)-1-(Prop-2-ynyl)piperidine-3-carboxamide

The product is obtained according to the procedure of Example 3 using the compound of Preparation 55 instead of the compound of Preparation 7.

Flash chromatography over silica gel (cyclohexane/AcOEt:2/8, then AcOEt) allows 550 mg of the expected product to be obtained.

Melting point: 154° C.
Elemental microanalyses:

| | C % | H % | N % |
|---|---|---|---|
| Calculated: | 72.57 | 6.81 | 14.94 |
| Found: | 72.38 | 6.97 | 14.78 |

Infrared (ν_{cm-1}): 3232 (ν≡C—H); 3050 (v═C—H); 2946; 2913 (νC—H); 2118 (νC≡C); 1698 (νC═O); 1614 (νC═C); 1569 (δN—H); 1475; 1458 (δC—H); 1368; 1340 (νC—N).

### EXAMPLE 65

### (±)—N-(Indol-1-yl)-1-[4-(piperidin-1-yl)but-2-en-1-yl]piperidine-3-carboxamide

To a solution of 140 mg of the compound of Preparation 2 in 4 ml of anhydrous dichloromethane, which solution has been cooled to −20° C., there is added 0.97 ml of 2M trimethylaluminium in hexane. After 1 hour 30 minutes without controlling the temperature, a solution of 260 mg of the compound of Preparation 56 in 2 ml of anhydrous dichloromethane is added at ambient temperature. The reaction mixture is heated at reflux for 12 hours; dichloromethane is added, and the mixture is then poured into 20% (w/v) aqueous sodium hydroxide solution. The organic phase is separated off and the aqueous phase is extracted with dichloromethane. The combined organic phases are dried over sodium sulphate, filtered and then evaporated under reduced pressure. Flash chromatography over silica gel (CH₂Cl₂/CH₃OH: 9:1, then 8/2) allows 310 mg of an impure fraction of the expected compound to be obtained. The solid is dissolved in 6 ml of a mixture of THF/H₂O (2:1). 50 mg of LiOH.H₂O are added at ambient temperature; the suspension is stirred for 12 hours. Dichloromethane and water are added to the reaction mixture. The organic phase is separated off and the aqueous phase is extracted with dichloromethane. The combined organic phases are dried over sodium sulphate, filtered and evaporated under reduced pressure. Flash chromatography over silica gel (CH₂Cl₂CH₃OH:9/1, then 8/2) allows 180 mg of the expected product to be obtained.

Melting point: 116° C.
Elemental microanalyses:

| | C % | H % | N % |
|---|---|---|---|
| Calculated: | 70.92 | 8.54 | 14.38 |
| Found: | 71.15 | 8.50 | 14.30 |

Infrared (ν_{cm-1}): 3166 (νN—H); 2999; 2934; 2854 (νC—H); 2798; 2754 (νN—CH₂); 1667 (νC═O); 1539 (δN—H); 1459 (δC—H); 1367 (νC—N).

### EXAMPLE 66

### (R or S) (−)-N-(Indol-1-yl)-1-[2-(piperidin-1-yl)ethyl]piperidine-3-carboxamide enantiomer 1

This compound is obtained by separation of the two enantiomers of the compound of Example 24, carried out by semi-preparative chiral HPLC (Daicel Chiralpak AD, 1×25 cm) under the following conditions: eluant: hexane (+0.1% TEA)/EtOH(+0.1% TEA): 96/4; flow rate: 4.7 ml/min.

Retention time: 17 min; purity 100%;

[α]_{D}²⁹=−15° (c=1.0; MeOH).

### EXAMPLE 67

### (R or S) (±)—N-(Indol-1-yl)-1-[2-(piperidin-1-yl)ethyl]piperidine-3-carboxamide enantiomer 2

This compound is obtained by separation of the two enantiomers of the compound of Example 24, carried out by semi-preparative chiral HPLC (Daicel Chiralpak AD, 1×25 cm) under the following conditions: eluant: hexane (+0.1% TEA)/EtOH(+0.1% TEA): 96/4; flow rate: 4.7 ml/min.

Retention time: 13 min; purity 100%;

[α]_{D}²⁹=+13° (c=1.0; MeOH).

### Pharmacological Study of Compounds of the Invention

### Example A

### Induction Of Tyrosine Hydroxylase

A search is made among the compounds for those which are capable of bringing about an increase in the tyrosine hydroxylase (TH) protein in the locus coeruleus (LC), substantia nigra (SN) and ventral tegmental area (VTA) of the brain of the Balb/C mouse. The animals used are consanguinal male mice of the pure Balb/C strain (Charles River Laboratories) aged 6 weeks at the time of treatment.

The mice are given a single injection, by the intraperitoneal route, of the compound under test, dissolved in 0.04M HCl solution (corresponding control: 0.04M HCl), if the compound is sufficiently soluble, or in olive oil 90%/DMSO 10% (corresponding control: olive oil 90%/DMSO 10%) for compounds that are insoluble in an aqueous medium. The volume injected is 100 μl. The test groups comprise 10 animals.

Three days after the injection of each compound, all the animals are sacrificed by decapitation. The brains are removed and then frozen in an isopentane solution at −30° C. for 45 seconds and subsequently stored at −80° C.

Serial coronal sections 20 microns thick are then taken using a cryomicrotome. Each structure of interest is then sampled along the posterior-anterior axis of the brain by taking sections at 80-micron spacings in the case of the LC and at 80- or 160-micron spacings in the case of the VTA and SN. The limits of those structures are those described in the atlas of Franklin and Paxinos. The total proteins of each of those sections are transferred by direct collection of the sections to a nitrocellulose filter (Millipore). The amounts of TH present in each sample are measured by immunochemistry, fluorimetric detection and image analysis.

### Results

The results for TH induction in the LC are given in Table I below.

TABLE I
Measurement of the amount of TH in the various LC sections, numbered from 1 to 11
in the posterior-to-anterior direction, after i.p. administration (20 mg/kg)
The results are expressed in %, relative to the mean value of the control group¹
(6 ≦ n ≦ 9)

| | % | | | | | | | | | | | |
|---|---|---|---|---|---|---|---|---|---|---|---|---|
| | 1 | 2 | 3 | 4 | 5 | 6 | 7 | 8 | 9 | 10 | 11 | LC, total |
| Ex. 1 | 57 | 72 | 128 | 128 | 158* | 152* | 131 | 107 | 106 | 95 | 55 | 121 |
| Ex. 7 | 113 | 139* | 112 | 119 | 138** | 122 | 141** | 131* | 96 | 125 | 106 | 123** |
| Ex. 12 | 57 | 70 | 80 | 110 | 114 | 92 | 95 | 82 | 98 | 94 | 26 | 90 |
| Ex. 22 | 79 | 101 | 112 | 98 | 108 | 113 | 146** | 120 | 135* | 179** | 145* | 119** |
| Ex. 24 | 165* | 157* | 120 | 127 | 145* | 131** | 139* | 116 | 139 | 96 | 94 | 129** |
| Ex. 26 | 152* | 98 | 136** | 107 | 154** | 133* | 127* | 96 | 132 | 110 | 89 | 121** |
| Ex. 47 | 232** | 138* | 176** | 179** | 172** | 169** | 153** | 140** | 84* | 88 | 127 | 147** |

*0.01 < p < 0.05
**p < 0.01
¹animals treated with the same carrier

The results for TH induction in the SN and VTA are given in Table II. They are expressed in %, relative to the mean value of the control group (6≦n≦9).

TABLE II

| | SN | VTA |
|---|---|---|
| Ex. 1 | 155** | 125 |
| Ex. 12 | 122* | 95 |
| Ex. 15 | 91 | 88* |
| Ex. 7 | 88* | 94 |

*0.01 < p < 0.05
**p < 0.01

The compound of Example 1 markedly induces TH in the medial LC (sections 4, 5 and 6) but also in the VTA and SN.

Other compounds induce TH in the SN without increasing expression of the protein in the LC and VTA (Example 12).

Others induce TH only in the LC (Example 7).

### Example B

### Affinity For Receptors

The affinity for receptors is determined according to customary methods of relations between the specific ligand and the receptor, which may be of animal origin or a human recombinant. The affinity was determined by the method of displacement of the labelled specific ligand by the compound under test and expressed by the dissociation constant KI. The receptor affinity was accordingly studied for 28 conventional receptors. The study shows that the TH induction observed does not proceed by way of affinity for receptors customarily affected by psychotropic compounds, such as alpha adrenergic receptors (type α₂), 5HT receptors (type 5HT2A) or dopaminergic receptors (types D₁ et D₂).

Some compounds exhibit an affinity for sigma (σ) receptors (ligand: haloperidol) or muscarinic (M) receptors that is not insignificant.

TABLE III

| % inhibition | α₂ | | 5HT2A | | D₁ | | D₂ | | M | | σ | |
|---|---|---|---|---|---|---|---|---|---|---|---|---|
| concentration (M) | 10⁻⁷ | 10⁻⁵ | 10⁻⁷ | 10⁻⁵ | 10⁻⁷ | 10⁻⁵ | 10⁻⁷ | 10⁻⁵ | 10⁻⁷ | 10⁻⁵ | 10⁻⁷ | 10⁻⁵ |
| Example 1 | — | — | — | — | — | — | — | — | — | 23 | — | 44 |
| Example 7 | — | — | — | — | — | — | — | — | — | — | — | — |
| Example 12 | — | — | — | — | — | — | — | — | — | 51 | 18 | 85 |
| Example 25 | — | — | 12 | 14 | — | — | — | — | — | 27 | 17 | 100 |
| Example 47 | — | — | 10 | 15 | — | — | — | — | 19 | 10 | — | 67 |

### Example C

### Predicted Crossing of the Blood-Brain Barrier (BBB)

The substances are incubated at 20 μM in the upper compartment of a double container, the upper compartment being separated from the lower compartment either solely by a polycarbonate filter or by the same filter covered with confluent endothelial cells from bovine capillaries. Evaluation of the permeability kinetics is carried out by means of LC-MS-MS quantification of the unchanged substance in the lower compartment after 10, 20, 30, 40 and 60 minutes.

The compounds tested generally exhibit a high degree of crossing of the BBB, which promotes access to the neurological target. The results are given in the form of the categories: high, intermediate, low. Accordingly, Examples 1, 7 and 24 exhibit a high degree of crossing of the BBB.

### Example D

### Predicted Metabolic Stability

The predicted metabolic stability is tested by incubation of the compounds at a concentration of 10⁻⁷M in the presence of mouse, rat or human hepatic microsomes (0.33 mg of prot/ml). After addition of NADPH (nicotinamide adenine dinucleotide phosphate, reduced form), samples are taken at 0, 5, 15, 30 and 60 minutes. The enzymatic reaction is stopped using methanol (V/V). The protein is precipitated by centrifugation and the supernatant is analysed by LC-MS-MS.

Good metabolic stability of the compounds makes it possible to envisage treatment per os.

TABLE IV
% metabolic stability predicted using hepatic microsomes

| | Mouse | Rat | Human |
|---|---|---|---|
| Example 1 | 40 | — | 70 |
| Example 25 | 85 | — | 63 |
| Example 47 | 53 | 87 | 78 |
| Example X (41319) | 17 | — | 85 |

### Example E

### Pharmaceutical Composition

Preparation formula for 1000 tablets each containing a dose of 10 mg

| | |
|---|---|
| Compound of Example 1 | 10 g |
| Hydroxypropylcellulose | 2 g |
| Wheat starch | 10 g |
| Lactose | 100 g |
| Magnesium stearate | 3 g |
| Talc | 3 g |

1-21. (canceled)

22. A compound selected from those of formula (I): 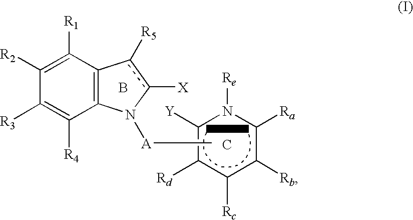
wherein:
A represents a divalent radical 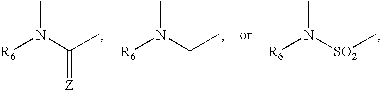
wherein:
Z represents an oxygen atom or sulphur atom,
R₆ represents
a hydrogen atom;
a linear or branched (C₁-C₆)alkyl group; C(O)-AA wherein AA represents an amino acid radical; a linear or branched (C₁-C₆)alkoxy-carbonyl group; CHR′—O—C(O)—R″ wherein R′ represents a hydrogen atom or a linear or branched (C₁-C₆)alkyl group and R″ represents a linear or branched (C₁-C₆)alkyl group;
a linear or branched (C₂-C₆)alkenyl group; an aryl group; an aryl-(C₁-C₆)alkyl group in which the alkyl moiety is linear or branched; a linear or branched (C₁-C₆)polyhaloalkyl group;
or a linear or branched (C₁-C₆)alkyl chain substituted by one or more halogen atoms, hydroxy groups, linear or branched (C₁-C₆)alkoxy groups, or amino groups wherein the amino groups may be optionally substituted by one or two identical or different, linear or branched (C₁-C₆)alkyl groups,
in the ring B [symbol]
represents a single bond or a double bond,
in the ring C [symbol]
represents a single bond or a double bond, the ring C containing, at most, only one double bond,
R₁, R₂, R₃ and R₄, which may be the same or different, each independently of the others, represent:
a hydrogen or halogen atom;
a linear or branched (C₁-C₆)alkyl group; a linear or branched (C₁-C₆)alkoxy group; a hydroxy group; a cyano group; a nitro group; a linear or branched (C₁-C₆)polyhaloalkyl group; an amino group wherein the amino group may be optionally substituted by one or two linear or branched (C₁-C₆)alkyl and/or linear or branched (C₂-C₆)alkenyl groups, which may be the same or different;
or a linear or branched (C₁-C₆)alkyl chain substituted by one or more halogen atoms, hydroxy groups, linear or branched (C₁-C₆)alkoxy groups, or amino groups wherein the amino groups may be optionally substituted by one or two identical or different, linear or branched (C₁-C₆)alkyl groups,
R₅ represents
a hydrogen atom;
a linear or branched (C₁-C₆)alkyl group; an aminoalkyl group in which the alkyl moiety is a linear or branched chain of 1 to 6 carbon atoms; or a linear or branched (C₁-C₆)-hydroxyalkyl group,
X and Y, which may be the same or different, each independently of the other, represent:
a hydrogen atom or a linear or branched (C₁-C₆)alkyl group,
Rₐ, R_{b}, R_{c} and R_{d}, which may be the same or different, each independently of the others, represent:
a hydrogen or halogen atom;
a linear or branched (C₁-C₆)alkyl group; a hydroxy group; a linear or branched (C₁-C₆)alkoxy group; a cyano group; a nitro group; a linear or branched (C₁-C₆)poly-haloalkyl group; an amino group wherein the amino group may be optionally substituted by one or two identical or different, linear or branched (C₁-C₆)alkyl groups;
or a linear or branched (C₁-C₆)alkyl chain substituted by one or more groups selected from halogen, hydroxy, linear or branched (C₁-C₆)alkoxy, and amino wherein the amino group may be optionally substituted by one or two identical or different, linear or branched (C₁-C₆)alkyl groups,
it being understood that when A is linked to the ring C at a carbon atom carrying one of the substituents Rₐ, R_{b}, R_{c}, R_{d} or Y and the linking carbon atom also carries a double bond, then the corresponding substituent Rₐ, R_{b}, R_{c}, R_{d} or Y is absent,
Rₑ represents:
a hydrogen atom;
a linear or branched (C₁-C₆)alkyl group; an aryl-(C₁-C₆)alkyl group in which the alkyl moiety is linear or branched; a linear or branched (C₂-C₆)alkenyl group; a linear or branched (C₂-C₆)alkynyl group; a linear or branched (C₁-C₆)alkyl chain substituted by one or more groups selected from hydroxy, amino wherein the amino group may be optionally substituted by one or two identical or different, linear or branched (C₁-C₆)alkyl groups, linear or branched (C₁-C₆)alkoxy, and NR₇R₈ wherein R₇ and R₈, together with the nitrogen atom carrying them, form an optionally substituted, 4- to 8-membered heterocycle optionally containing one or more double bonds within the heterocycle and optionally containing within the cyclic system a second hetero atom selected from an oxygen atom and a nitrogen atom; or a linear or branched (C₂-C₆)alkenyl chain substituted by one or more groups selected from hydroxy, amino wherein the amino group may be optionally substituted by one or two identical or different, linear or branched (C₁-C₆)alkyl groups, linear or branched (C₁-C₆)alkoxy, and NR₇R₈ wherein R₇ and R₈, together with the nitrogen atom carrying them, form an optionally substituted, 4- to 8-membered heterocycle optionally containing one or more double bonds within the heterocycle and optionally containing within the cyclic system a second hetero atom selected from an oxygen atom and a nitrogen atom; or a linear or branched (C₂-C₆)alkynyl chain substituted by one or more groups selected from hydroxy, amino wherein the amino group may be optionally substituted by one or two identical or different, linear or branched (C₁-C₆)alkyl groups, linear or branched (C₁-C₆)alkoxy, and NR₇R₈ wherein R₇ and R₈, together with the nitrogen atom carrying them, form an optionally substituted, 4- to 8-membered heterocycle optionally containing one or more double bonds within the heterocycle and optionally containing within the cyclic system a second hetero atom selected from an oxygen atom and a nitrogen atom,
its enantiomers, diastereoisomers, and N-oxides, and addition salts thereof with a pharmaceutically acceptable acid or base,
it being understood that:
the 4- to 8-membered heterocycle optionally containing one or more double bonds within the heterocycle and optionally containing within the cyclic system a second hetero atom selected from an oxygen atom and a nitrogen atom, may be optionally substituted by one or more identical or different groups selected from linear or branched (C₁-C₆)alkyl; linear or branched (C₁-C₆)hydroxyalkyl; linear or branched (C₁-C₆)alkoxy-(C₁-C₆)alkyl; CO₂Rᵥ, CO₂—R_{w}—NRᵥR′ᵥ, or CO₂—R_{w}—ORᵥ, wherein Rᵥ represents a hydrogen atom or a linear or branched (C₁-C₆)alkyl group, R′ᵥ represents a hydrogen atom or a linear or branched (C₁-C₆)alkyl group, and R_{w}, represents a linear or branched (C₁-C₆)alkylene chain; aryl; aryloxycarbonyl; linear or branched aryl-(C₁-C₆)alkoxy-carbonyl; optionally substituted cycloalkyl; optionally substituted cycloalkylalkyl; optionally substituted heterocycloalkyl; optionally substituted heterocycloalkylalkyl; and aminoalkyl wherein the alkyl moiety is a linear or branched chain of 1 to 6 carbon atoms and the amino moiety may be optionally substituted by one or two identical or different, linear or branched (C₁-C₆)alkyl groups,
aryl means a phenyl or naphthyl group, wherein the phenyl or naphthyl group may be optionally substituted by one or more halogen atoms, nitro, amino, linear or branched (C₁-C₆)alkyl or linear or branched (C₁-C₆)alkoxy groups,
cycloalkyl means a saturated, 4- to 8-membered, monocyclic group,
cycloalkylalkyl means a cycloalkyl-alkyl group wherein the alkyl group denotes a linear or branched chain of 1 to 6 carbon atoms and the cycloalkyl group denotes a saturated, 4- to 8-membered, monocyclic group,
heterocycloalkyl means a saturated, 4- to 8-membered, monocyclic group containing 1 or 2 hetero atoms selected from nitrogen, oxygen and sulphur,
heterocycloalkylalkyl means a heterocycloalkyl-alkyl group wherein the alkyl group denotes a linear or branched chain of 1 to 6 carbon atoms and the heterocycloalkyl group denotes a saturated, 4- to 8-membered, monocyclic group containing 1 or 2 hetero atoms selected from nitrogen, oxygen and sulphur,
the expression “optionally substituted” when referring to the groups cycloalkyl, cycloalkylalkyl, heterocycloalkyl and heterocycloalkylalkyl means that these groups may be substituted by one or more identical or different substituents selected from linear or branched (C₁-C₆)alkyl; linear or branched (C₁-C₆)hydroxyalkyl; linear or branched (C₁-C₆)alkoxy-(C₁-C₆)alkyl; carboxy; linear or branched (C₁-C₆)alkoxy-carbonyl; and aminoalkyl wherein the alkyl moiety is a linear or branched chain of 1 to 6 carbon atoms and the amino moiety may be optionally substituted by one or two identical or different, linear or branched (C₁-C₆)alkyl groups,
an amino acid radical means a radical selected from alanyl, arginyl, asparaginyl, α-aspartyl, cysteinyl, α-glutamyl, glutaminyl, glycyl, histidyl, isoleucyl, leucyl, lysyl, methionyl, phenylalanyl, prolyl, seryl, threonyl, tryptophyl, tyrosyl and valyl.

23. A compound of claim 22, wherein A represents a divalent radical: 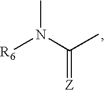
wherein Z represents an oxygen atom, its enantiomers and diastereoisomers, and addition salts thereof with a pharmaceutically acceptable acid or base.

24. A compound of claim 22, wherein R₆ represents a hydrogen atom, its enantiomers, diastereoisomers, and N-oxides, and addition salts thereof with a pharmaceutically acceptable acid or base.

25. A compound of claim 22, wherein R₁, R₂, R₃ and R₄ represent a hydrogen atom, a halogen atom or a linear or branched (C₁-C₆)alkoxy group, its enantiomers, diastereoisomers, and N-oxides, and addition salts thereof with a pharmaceutically acceptable acid or base.

26. A compound of claim 22, wherein R₅ represents a hydrogen atom or a linear or branched (C₁-C₆)alkyl group, its enantiomers, diastereoisomers, and N-oxides, and addition salts thereof with a pharmaceutically acceptable acid or base.

27. A compound of claim 22, wherein X and Y represent a hydrogen atom or a linear or branched (C₁-C₆)alkyl group, its enantiomers, diastereoisomers, and N-oxides, and addition salts thereof with a pharmaceutically acceptable acid or base.

28. A compound of claim 22, wherein Rₐ, R_{b}, R_{c} and R_{d} represent a hydrogen atom, its enantiomers, diastereoisomers, and N-oxides, and addition salts thereof with a pharmaceutically acceptable acid or base.

29. A compound of claim 22, wherein Rₑ represents a hydrogen atom or a linear or branched (C₁-C₆)alkyl or linear or branched (C₂-C₆)alkenyl group, its enantiomers, diastereoisomers, and N-oxides, and addition salts thereof with a pharmaceutically acceptable acid or base.

30. A compound of claim 22, which is selected from those of formula (IA): 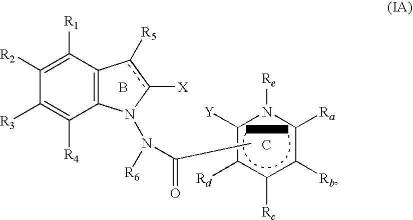
its enantiomers, diastereoisomers, and N-oxides, and addition salts thereof with a pharmaceutically acceptable acid or base.

31. A compound of claim 22, which is selected from those of formula (IB): 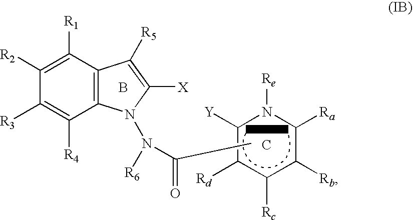
its enantiomers, diastereoisomers, and N-oxides, and addition salts thereof with a pharmaceutically acceptable acid or base.

32. A compound of claim 22, which is selected from those of formula (IC): 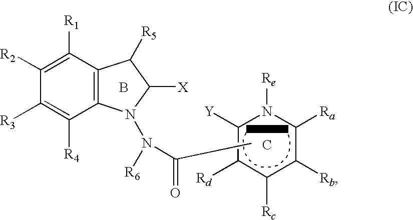
its enantiomers, diastereoisomers, and N-oxides, and addition salts thereof with a pharmaceutically acceptable acid or base.

33. A compound of claim 22, which is selected from those of formula (ID): 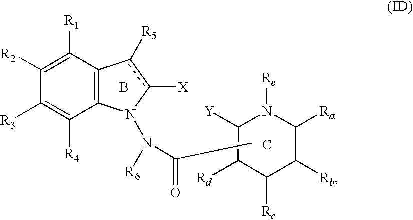
its enantiomers, diastereoisomers, and N-oxides, and addition salts thereof with a pharmaceutically acceptable acid or base.

34. A compound of claim 22, which is selected from those of formula (IE): 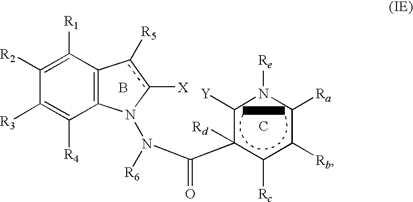
its enantiomers, diastereoisomers, and N-oxides, and addition salts thereof with a pharmaceutically acceptable acid or base.

35. A compound of claim 22, which is selected from those of formula (IF): 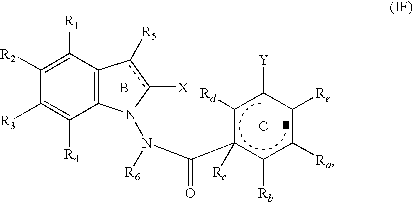
its enantiomers, diastereoisomers, and N-oxides, and addition salts thereof with a pharmaceutically acceptable acid or base.

36. A compound of claim 22, which is selected from those of formula (IG): 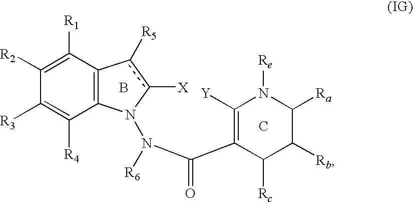
its enantiomers, diastereoisomers, and N-oxides, and addition salts thereof with a pharmaceutically acceptable acid or base.

37. A compound of claim 22, which is selected from those of formula (IH): 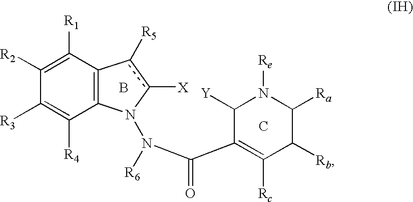
its enantiomers, diastereoisomers, and N-oxides, and addition salts thereof with a pharmaceutically acceptable acid or base.

38. A compound of claim 22, which is selected from those of formula (IJ): 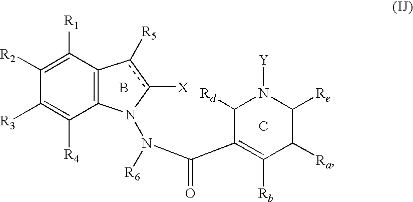
its enantiomers, diastereoisomers, and N-oxides, and also addition salts thereof with a pharmaceutically acceptable acid or base.

39. A compound of claim 22, which is selected from:
N-(1H-indol-1-yl)-1-methyl-1,2,5,6-tetrahydropyridine-3-carboxamide,
N-(2,3-dihydro-1H-indol-1-yl)-1-methyl-1,4,5,6-tetrahydropyridine-3-carboxamide,
N-(5-fluoro-1H-indol-1-yl)-1-methyl-1,2,5,6-tetrahydropyridine-3-carboxamide,
N-(2,3-dihydro-1H-indol-1-yl)-1-methyl-1,4,5,6-tetrahydropyridine-3-carboxamide,
1-[2-(dimethylamino)ethyl]-N-(1H-indol-1-yl)-1,2,5,6-tetrahydropyridine-3-carboxamide,
N-(1H-indol-1-yl)-1-[2-(4-methyl-1-piperazinyl)ethyl]-3-piperidinecarboxamide,
N-(5-chloro-1H-indol-1-yl)-1-(2-hydroxyethyl)-1,4,5,6-tetrahydropyridine-3-carboxamide,
tert-butyl 4-(2-{3-[(1H-indol-1-ylamino)carbonyl]-1-piperidyl}ethyl)piperazine-1-carboxylate,
1-[3-(dimethylammonium)propyl]-3-[(1H-indol-1-ylamino)carbonyl]piperidinium,
N-(1H-indol-1-yl)-1-[3-(1-piperidyl)propyl]-3-piperidinecarboxamide,
N-(1H-indol-1-yl)-1-[3-(4-methyl-1-piperazinyl)propyl]-3-piperidinecarboxamide,
N-(indol-1-yl)-1-(2-piperidin-1-yl-ethyl)-1,2,5,6-tetrahydropyridine-3-carboxamide,
(±)—N-(indol-1-yl)-1-[2-[4-(1-methylpiperidin-4-yl)piperazin-1-yl)]ethyl]piperidine-3-carboxamide,
(±)—N-(indol-1-yl)-1-[3-[4-(2-hydroxyethyl)piperazin-1-yl)]propyl]piperidine-3-carboxamide,
(±)—N-(indol-1-yl)-1-[4-(4-methylpiperazin-1-yl)butyl]piperidine-3-carboxamide,
(±)—N-(indol-1-yl)-1-allylpiperidine-3-carboxamide,
(±)—N-(indol-1-yl)-1-[4-(piperidin-1-yl)but-2-en-1-yl]piperidine-3-carboxamide,
(R or S) (−)-N-(indol-1-yl)-1-[2-(piperidin-1-yl)ethyl]piperidine-3-carboxamide enantiomer 1,
(R or S) (±)—N-(indol-1-yl)-1-[2-(piperidin-1-yl)ethyl]piperidine-3-carboxamide enantiomer 2,
its enantiomers, diastereoisomers, and N-oxides, and addition salts thereof with a pharmaceutically acceptable acid or base.

40. A pharmaceutical composition comprising as active ingredient at least one compound of claim 22 in combination with one or more inert, non-toxic, pharmaceutically acceptable excipients or carriers.

41. A method of treating a living animal body, including a human, afflicted with a condition selected from depression, anxiety, disorders of memory associated with aging and/or neurodegenerative diseases, stress, comprising the step of administering to the living animal body, including a human, an amount of a compound of claim 22 which is effective for treatment of the condition.

42. A method of treating a living animal body, including a human, afflicted with Parkinson's disease, comprising the step of administering to the living animal body, including a human, an amount of a compound of claim 22 which is effective for the palliative treatment of the condition.
